(19) United States
(12) Patent Application Publication
(10) Pub. No.: US 2025/0017578 A1
(43) Pub. Date: Jan. 16, 2025

(54) SYSTEMS AND METHODS FOR SHUNTING AND DEVICE IMPLANTATION

(71) Applicant: EDWARDS LIFESCIENCES CORPORATION, Irvine, CA (US)

(72) Inventors: Anthony Ciro Vrba, San Clemente, CA (US); Scott Louis Pool, Laguna Hills, CA (US)

(21) Appl. No.: 18/898,337

(22) Filed: Sep. 26, 2024

Related U.S. Application Data
(63) Continuation of application No. PCT/US2023/017301, filed on Apr. 3, 2023.
(60) Provisional application No. 63/327,056, filed on Apr. 4, 2022.

(51) Int. Cl. A61B 17/00; A61B 90/00

(52) U.S. Cl. CPC A61B 17/0057; A61B 90/39; A61B 2017/00477; A61B 2017/00575; A61B 2017/00597; A61B 2017/00606; A61B 2017/00619; A61B 2017/00623; A61B 2017/00946; A61B 2090/3966

(57) ABSTRACT

Devices, systems and methods for shunting, plugging, and device installation via a cinching mechanism are provided. In some instances, a cinching system incorporates two rings and a cord stitched along and between the two rings. In some instances, the two termini of the cord can be pulled to provide a cinching mechanism. In some instances, the cinching system is sheathed within a transcatheter delivery system, which can used to deliver the cinching system to a target site.

### CROSS REFERENCE TO RELATED APPLICATIONS

This application is a continuation of International Patent Application No. PCT/US2023/017301, filed Apr. 3, 2023, which claims the benefit of U.S. Provisional Application No. 63/327,056, filed Apr. 4, 2022, the disclosures of which are hereby incorporated by reference in their entirety for all purposes.

### TECHNICAL FIELD

The disclosure is generally directed to systems and methods for shunting and device implantation within the cardiovascular system, and more specifically systems and methods utilizing a cinching mechanism for shunting and device implantation.

### BACKGROUND

Various disorders of the cardiovascular system can be treated by a shunt that allows for blood flow from one area to another. Generally, a shunt is an aperture and passageway to allow blood to travel through tissue, such as a vascular wall or a heart chamber wall. For instance, to reduce the symptoms and slow the progression of heart failure, a shunt can be implanted in the wall of the left atrium to allow blood flow from the left atrium into the right atrium via the coronary sinus, providing decompression of the left atrium.

### SUMMARY OF THE DISCLOSURE

The description includes systems and methods for shunting within the body, such as a shunt within the cardiovascular system. In many instances, a shunt is created within a tissue wall of the cardiovascular system to allow blood flow from one area to another; the shunt is secured via a system of cinch rings. In several instances, a system of cinch rings comprises two cinch rings and a cord, the cord interlinking between the two rings such that when the cord is tightened, the two rings pulled together. In many instances, a distal ring of the system is capable of being installed on a distal side of the shunt and surrounding tissue wall; a proximal ring of the system is capable of being installed on a proximal side of the shunt and surrounding tissue wall; and a cord interlinking the distal and proximal rings provides a cinching mechanism such that the two rings are capable of being pulled together and tightened against the distal and proximal sides of the tissue wall, securing the shunt and holding the punctured tissue open.

The description includes systems and methods for occluding an aperture within the body, such as a coronary arteriovenous fistula occurring within the cardiovascular system. In many instances, a plug is provided to occlude a fistula of the cardiovascular system to prevent blood flow through the fistula; the plug comprises a system of cinch rings. In several instances, the system of cinch rings comprises two cinch rings and a cord, at least one of the rings being closed via a cover, and the cord interlinking between the two rings such that when the cord is tightened, the two rings pulled together. In many instances, a distal ring of the system is capable of being installed on a distal side of the fistula and surrounding tissue wall; a proximal ring of the system is capable of being installed on a proximal side of the fistula and surrounding tissue wall; and a cord interlinking the distal and proximal rings provides a cinching mechanism such that the two rings are capable of being pulled together and tightened against the distal and proximal sides of the tissue wall, occluding the fistula.

The description includes systems and methods for implanting a device within a tissue wall of the body, such as a tissue wall within the vasculature system. In many instances, the device is secured to the tissue wall via a system of cinch rings. In several instances, the system of cinch rings comprises two cinch rings and a cord, and the cord interlinking between the two rings such that when the cord is tightened, the two rings pulled together. In many instances, the device secured or in connection with at least one of the rings. In several instances, a distal ring of the system is capable of being installed on a distal side of a tissue wall; a proximal ring of the system is capable of being installed on a proximal side of the tissue wall; and a cord interlinking the distal and proximal rings provides a cinching mechanism such that the two rings are capable of being pulled together and tightened against the distal and proximal sides of the tissue wall, securing the device to the tissue wall. In many instances, the device is a telemetric sensor. In various instances, the telemetric sensor is a pressure sensor, a temperature sensor, a flow rate sensor, oxygen saturation sensor, loop recorder (cardiac monitor), any other hemodynamic sensor, or any combination of sensors.

In some implementations, a system is for cinching two rings together. The system comprises a first ring comprising an outer face, an inner face, and a plurality of stitch holes. The system further comprises a second ring comprising an outer face, an inner face, and a plurality of stitch holes. The system further comprises a set of one or more cords that is stitched along and between the first ring and the second ring via the plurality of stitch holes of the first and second rings. Each of the two termini of each cord extends out from the external face of the first ring via a stitch hole of the first ring.

In some implementations, the system has a cinching mechanism capability that moves the first ring and the second ring towards one another when the set of one or more cords is tightened by concurrently pulling the two termini of each cord in a direction away from the external face of the first ring.

In some implementations, the system further comprises a means for providing a friction force against the first ring when tightening the cord by concurrently pulling the two termini of the cord in a direction away from the external face of the first ring.

In some implementations, the means for providing a friction force are a set of two tubes, each tube surrounding a portion of the cord that extends out from the external face of the first ring via a stitch hole of the first ring.

In some implementations, the set of one or more cords is a single cord.

In some implementations, the set of one or more cords is a plurality of cords.

In some implementations, a cinch ring system is for providing a shunt within the body of a patient. The cinch ring system comprises a proximal ring comprising a proximal face, a distal face, and a plurality of stitch holes. The cinch ring further comprises a distal ring comprising a proximal face, a distal face, and a plurality of stitch holes. The cinch ring further comprises a cord that is stitched along and between the proximal ring and the distal ring via the plurality of stitch holes of the proximal and distal rings. Each of the two termini of the cord extend out from the proximal face of the proximal ring via a stitch hole of the proximal ring.

In some implementations, the cinch ring system has a cinching mechanism capability that moves the proximal ring and the distal ring towards one another when the cord is tightened by concurrently pulling the two termini of the cord in a proximal direction away from the proximal face of the proximal ring.

In some implementations, the proximal ring or the distal ring contain at least one radiopaque marker.

In some implementations, the proximal ring and the distal ring each contain a plurality of radiopaque markers.

In some implementations, the plurality of radiopaque markers of the proximal ring are in an orientation that mirrors the plurality of radiopaque markers of the distal ring when the proximal ring and the distal ring are provided in parallel planes.

In some implementations, the plurality of radiopaque markers of the proximal ring and the distal ring are each provided in an orientation relative to one another such that the opaque markers lack mirrored symmetry along one central axis of the ring.

In some implementations, at least one ring of the proximal ring and the distal rings comprises a set of one or more protrusions extending outwardly from the ring.

In some implementations, at least one ring of the proximal ring and the distal rings comprises a set of one or more windows.

In some implementations, at least one stitch hole of the proximal ring or the distal ring incorporates a cord grip.

In some implementations, each stitch hole of the proximal ring in which the two termini of the cord extend out from incorporates a cord grip.

In some implementations, at least one ring of the proximal ring and the distal ring incorporates a sensor.

In some implementations, the proximal ring and the distal ring are each provided in an elongated shape.

In some implementations, the proximal ring and the distal ring are each capable of folding or curving inward such that it is capable of being sheathed within a transcatheter capable of delivery via the circulatory system.

In some implementations, the proximal ring and the distal ring each have a central aperture with an area that is greater than the cross-sectional area of the transcatheter.

In some implementations, the proximal ring and the distal ring are each composed of a biocompatible and malleable material.

In some implementations, the cord is composed of a biocompatible material and is provided as a wire, a monofilament, or a twine.

In some implementations, a cinch ring is for providing a plug within the body of a patient. The cinch ring system comprises a proximal ring comprising a proximal face, a distal face, and a plurality of stitch holes. The cinch ring system further comprises a distal ring comprising a proximal face, a distal face, and a plurality of stitch holes, wherein a cover spans the central aperture of at least one ring of the proximal ring and the distal ring. The cinch ring system further comprises a cord that is stitched along and between the proximal ring and the distal ring via the plurality of stitch holes of the proximal and distal rings. Each of the two termini of the cord extend out from the proximal face of the proximal ring via a stitch hole of the proximal ring.

In some implementations, the system has a cinching mechanism capability that moves the proximal ring and the distal ring towards one another when the cord is tightened by concurrently pulling the two termini of the cord in a proximal direction away from the proximal face of the proximal ring.

In some implementations, a cover spans the central aperture of each ring of the proximal ring and the distal ring.

In some implementations, wherein each cover incorporates a through hole or slit.

In some implementations, the through hole or slit is scalable.

In some implementations, the proximal ring or the distal ring contain at least one radiopaque marker.

In some implementations, at least one ring of the proximal ring and the distal rings comprises a set of one or more protrusions extending outwardly from the ring.

In some implementations, the proximal ring and the distal ring are each provided in an elongated shape.

In some implementations, the proximal ring and the distal ring are each capable of folding in or curving in itself such that it is capable of being sheathed within a transcatheter capable of delivery via the circulatory system.

In some implementations, the proximal ring and the distal ring are each composed of a biocompatible and malleable material.

In some implementations, the cord is composed of a biocompatible material and is provided as a wire, a monofilament, or a twine.

In some implementations, a delivery system is for delivery a cinch ring system to a target site within the body of a patient. The delivery system comprises a catheter comprising an outer sheath and an inner sheath. The delivery system further comprises a cinch ring system. The cinch ring system comprises a proximal ring comprising a proximal face, a distal face, and a plurality of stitch holes. The cinch ring system further comprises a distal ring comprising a proximal face, a distal face, and a plurality of stitch holes. The cinch ring system further comprises a cord that is stitched along and between the proximal ring and the distal ring via the plurality of stitch holes of the proximal and distal rings. The cinch ring system has a cinching mechanism. The cinch ring system is contained within the outer sheath and is in connection with the inner sheath of the catheter.

In some implementations, the proximal ring and the distal ring are each in a deliverable conformation within the outer sheath of the catheter.

In some implementations, the proximal ring and the distal ring are each an elongated shape that is folded or curved inwards.

In some implementations, the proximal ring and the distal ring are spaced apart from one another to facilitate traversing through the vasculature system of a patient.

In some implementations, the cinch ring system is in connection with the inner sheath via a set of one or more release wires.

In some implementations, the set of one or more release wires comprises at least three release wires. Each release wire provides an attachment point to connect the cinch ring system with the inner sheath.

In some implementations, a first release wire provides a first attachment to the distal ring. A second release wire provides an attachment to the proximal ring. A third release wire provides a second attachment to the distal ring.

In some implementations, the inner sheath and the outer sheath are each independently capable of moving in a proximal direction or in a distal direction.

In some implementations, the connection of the cinch ring system to the inner sheath allows for the cinch ring system to be sheathed and unsheathed within the outer sheath.

In some implementations, a cover spans the central aperture of each ring of the proximal ring and the distal ring. Each cover incorporates a through hole or slit, and wherein the inner sheath traverses through each through hole or slit.

In some implementations, a method is for cinching a cinch ring system. The method comprises providing a cinch ring system. The cinch ring system comprises a first ring comprising an outer face, an inner face, and a plurality of stitch holes. The cinch ring system further comprises a distal ring comprising a proximal face, a distal face, and a plurality of stitch holes. The cinch ring system further comprises a cord that is stitched along and between the proximal ring and the distal ring via the plurality of stitch holes of the proximal and distal rings. Each of the two termini of the cord extend out from the proximal face of the proximal ring via a stitch hole of the proximal ring. The cinch ring system is contained within the outer sheath and is in connection with the inner sheath of the catheter. The method further comprises delivering the transcatheter delivery system to a tissue wall having an aperture. The method further comprises installing the cinch ring system to the tissue wall.

In some implementations, the installing of the cinch ring system to the tissue wall comprises releasing the distal ring on the distal side of the tissue wall. The installing of the cinch ring system to the tissue wall further comprises releasing the proximal ring on the proximal side of the tissue wall. At least a portion of the cord that is stitched between the distal ring and proximal ring traverses through the aperture of the tissue wall. The installing of the cinch ring system to the tissue wall further comprises cinching the cinch ring system such that the distal ring is in proximity to or in contact with the distal side of the tissue wall and the proximal ring is in proximity to or in contact with the proximal side of the tissue wall.

In some implementations, the cinch ring system is in connection with the inner sheath via a set of one or more release wires. At least one release wire of the set is in connection with the distal ring. The releasing of the distal ring on the distal side of the tissue wall comprises disconnecting the at least one release wire of the set that is in connection with the distal ring.

In some implementations, the cinch ring system is in connection with the inner sheath via a set of one or more release wires. At least one release wire of the set is in connection with the proximal ring. The releasing of the proximal ring on the proximal side of the tissue wall comprises disconnecting the at least one release wire of the set that is in connection with the proximal ring.

In some implementations, the cinching of the cinch ring system comprises pulling on the two termini of the cord in a proximal direction to tighten the cord.

In some implementations, the installing of the cinch ring system to the tissue wall further comprises securing the cord to keep the cord taut and the cinching ring system in place via constrictive forces.

In some implementations, the cord is secured using a cord grip.

In some implementations, the cord is secured by tying off the cord.

In some implementations, the proximal ring and the distal ring each contain a plurality of radiopaque markers. The method further comprises aligning the proximal ring and the distal ring via the plurality of radiopaque markers on each the proximal ring and the distal ring.

In some implementations, the plurality of radiopaque markers of the proximal ring are in an orientation that mirrors the plurality of radiopaque markers of the distal ring when the cinch ring system is installed on the tissue wall.

In some implementations, the proximal ring and the distal ring are each folded or curved inward when within the outer sheath.

In some implementations, installation of the cinch ring system to the tissue wall provides a shunt via the aperture in the tissue wall.

In some implementations, a cover spans a central aperture of at least one ring of the proximal ring and the distal ring, wherein installation of the cinch ring system to the tissue wall provides a plug of the aperture in the tissue wall.

In some implementations, the installation of the cinch ring system to the tissue wall further comprises sealing a through hole or slit in the cover.

In some implementations, delivering of the transcatheter delivery system is delivered via a transfemoral approach, a subclavian approach, a transapical approach, or a transaortic approach.

In some implementations, a method is for installing a cinch ring system on a nonliving simulation having a simulated tissue wall. The method comprises providing a nonliving tissue wall having an aperture. The nonliving tissue wall provides a simulation of a tissue wall within a patient. The method further comprises providing a transcatheter delivery system. The transcatheter delivery system comprises a catheter comprising an outer sheath and an inner sheath. The transcatheter delivery system further comprises a cinch ring system. The cinch ring system comprises a proximal ring comprising a proximal face, a distal face, and a plurality of stitch holes. The cinch ring system further comprises a distal ring comprising a proximal face, a distal face, and a plurality of stitch holes. The cinch ring system further comprises a cord that is stitched along and between the proximal ring and the distal ring via the plurality of stitch holes of the proximal and distal rings. Each of the two termini of the cord extend out from the proximal face of the proximal ring via a stitch hole of the proximal ring. The cinch ring system is contained within the outer sheath and is attached to the inner sheath of the catheter. The method further comprises releasing the distal ring on the distal side of the nonliving tissue wall. The method further comprises releasing the proximal ring on the proximal side of the nonliving tissue wall. At least a portion of the cord stitched between the distal ring and proximal ring traverses through the aperture of the nonliving tissue wall. The method further comprises pulling on the two termini of the cord in a proximal direction to tighten the cord such that the distal ring is in proximity to or in contact with the distal side of the nonliving tissue wall and the proximal ring is in proximity to or in contact with the proximal side of the nonliving tissue wall.

In some implementations, a cinch ring system is for installing a device within the body of a patient. The cinch ring system comprises a proximal ring comprising a proximal face, a distal face, a central aperture, and a plurality of stitch holes. The cinch ring system further comprises a distal ring comprising a proximal face, a distal face, a central aperture, and a plurality of stitch holes. The cinch ring system further comprises a cord that is stitched along and between the proximal ring and the distal ring via the plurality of stitch holes of the proximal and distal rings. Each of the two termini of the cord extend out from the proximal face of the proximal ring via a stitch hole of the proximal ring. The system has a cinching mechanism capability that moves the proximal ring and the distal ring towards one another when the cord is tightened by concurrently pulling the two termini of the cord in a proximal direction away from the proximal face of the proximal ring. The cinch ring system further comprises a device having a body that is in connection with the central aperture of the proximal ring or the central aperture of the distal ring. The proximal ring or the distal ring is capable of sliding along the body the device when the cinching mechanism is utilized.

In some implementations, the device is a sensor.

In some implementations, the sensor is a telemetric sensor.

In some implementations, the sensor is a hemodynamic sensor.

In some implementations, the sensor is a pressure sensor, a temperature sensor, a flow rate sensor, an oxygen saturation sensor, or a loop recorder.

In some implementations, at least one ring of the proximal ring and the distal rings comprises a set of one or more protrusions extending outwardly from the ring.

In some implementations, each protrusion of the set one or more protrusions has length between 1X and 5X of the internal diameter of proximal ring or the distal ring.

In some implementations, the proximal ring or the distal ring contain at least one radiopaque marker.

In some implementations, at least one ring of the proximal ring and the distal rings comprises a set of one or more windows.

In some implementations, at least one stitch hole of the proximal ring or the distal ring incorporates a cord grip.

In some implementations, each stitch hole of the proximal ring in which the two termini of the cord extend out from incorporates a cord grip.

In some implementations, the proximal ring and the distal ring have a total area that is less than a cross-sectional area of the transcatheter, wherein in the total area is exclusive of any extended protrusions.

In some implementations, the proximal ring and the distal ring each have a set of one or more extended protrusions. The extended protrusions are capable of being folded into a position perpendicular to the faces of the proximal ring and the distal ring such that the proximal ring and the distal ring are each capable of fitting within the cross-section area of the transcatheter.

In some implementations, the proximal ring and the distal ring are each composed of a biocompatible and malleable material.

In some implementations, the cord is composed of a biocompatible material and is provided as a wire, a monofilament, or a twine.

### BRIEF DESCRIPTION OF THE DRAWINGS

The description and claims will be more fully understood with reference to the following figures and schematics, which are presented as examples of the disclosure and should not be construed as a complete recitation of the scope of the disclosure.

FIGS. 1A to 6C provide various examples of a cinching system.

FIGS. 7A to 9B provide various examples of a cinching system installed on a tissue wall.

FIGS. 10A to 10E provide various examples of cinching system for device installation.

FIG. 11A and 11B provide an example of a transcatheter with a cinching system for device installation.

FIG. 12 provides an example of a cinching system with device installed on a tissue wall.

FIG. 13 provides an example of a transcatheter with a cinching system therein.

FIGS. 14A to 14C provide an example of sheathing a cinching system within a transcatheter.

FIGS. 15A to 15H provide an example of delivering and installing a cinching system onto a tissue wall.

### DETAILED DESCRIPTION

Turning now to the drawings, systems and devices that incorporate a cinching mechanism for shunting, plugging, and/or device securement, and methods of use thereof, are described. In several instances, systems and devices incorporate two or more rings and at least one cord that are combined such that the two rings are capable of being pulled together by pulling and tightening the cord, providing a cinching mechanism. In many instances, the cord of the cinching system is stitched or looped along each ring and between the two rings, providing an interconnection between the two rings. In several instances, at least one end of the cord can be pulled, which will result in tightening the stitching or looping along and between the rings, pulling the two rings together. Although the various depictions and descriptions of the cinching system are provided as utilizing two or more rings, any shape can be utilized in accordance with the various instances.

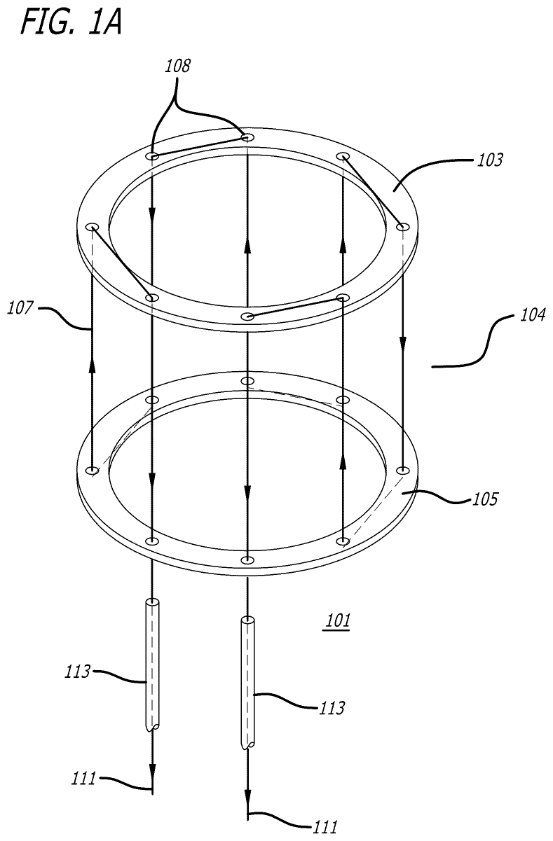

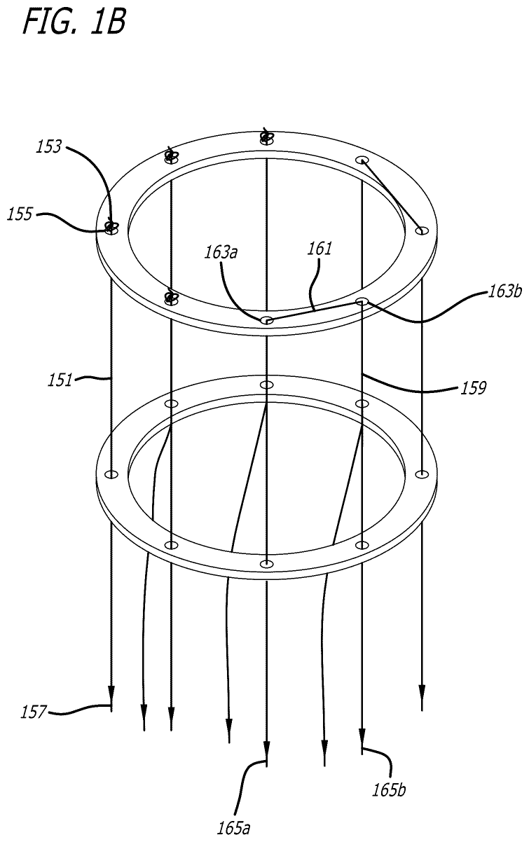

Provided in FIG. 1 is an example of cinching system 101. As shown in this example, cinching system 101 includes a distal ring 103 and a proximal ring 105 and a cord 107 that is stitched along and between the two rings, through a plurality of stitch holes 108. Each of the two rings are in parallel planes (or near parallel planes) such that inner face of distal ring 103 faces the inner face of proximal ring 105. The stitching of cord 107 connects the two rings, but can be initially provided loosely such that there is a central space 104 between the planes of the rings. Space 104 can vary, and the amount of space required between the two rings will depend on the application and procedure for installing the cinching system.

As shown, a single cord (cord 107) is utilized in the cinching system, with the two termini 111 of the cord being on the proximal side of proximal ring 105, which allows both ends to be pulled concurrently. Pulling cord ends 111 concurrently in the proximal direction tightens cord 107 and pulls distal ring 103 and proximal ring 105 together, narrowing central space 104. To assist in tightening cord 107, a set of tubes 113 surrounding the proximal portions of the cord can be utilized to provide an opposition force against proximal ring 105. To provide the force, the set of tubes 113 can be slid distally along cord 107 such that the distal ends of the tubes come into contact with the proximal face of proximal ring 105, creating a force as the cord ends 111 are pulled proximally. In some instances, when a cinching system is to be delivered and installed by a transcatheter system, a component of the transcatheter system can provide the opposition force for tightening the cord. In some instances using the transcatheter system, the outer sheath provides the force.

Although a single cord is described in the cinching system of FIG. 1A, multiple cords can be utilized, as shown in the examples provided within FIG. 1B. For instance, cord 151 is a single directional cord that has a distal terminus 153 tied off at a stitch hole 155 the distal end of the cinch ring system and a proximal terminus 157 that extends proximally and is used to tighten the cord and bring the two rings of the cinch ring system together. Cord 159 is a single stitched cord that has a distal stitch portion 161 that is stitched among two stitch holes 163a and 163b of the distal ring and two proximal termini 165a and 165b that each extend proximally and are used concurrently to tighten the cord and bring the two rings of the cinch ring system together. In some instances, utilization of multiple cords may be preferred, as the use of multiple cords allows for more local control in tightening the cinch ring system. For example, utilization of multiple cords allows just a portion of the cinch ring system (e.g., a right side) to be tightened while another portion of the cinch ring system (e.g., a left side) can remain loose, which may help installing a cinch ring system depending on the local contours of the site of installation.

And although a set of tubes is utilized to provide an opposition force against the proximal ring, the need for an opposition force is optional, such as when one of the rings is secured in place. Further, when an opposition force is desired, any object capable of providing the force as the cord is being tightened may be utilized in place of the set of tubes.

The described systems, devices, and methods, should not be construed as limiting in any way. Instead, the present disclosure is directed toward all novel and nonobvious features and aspects of the various disclosed examples, alone and in various combinations and sub-combinations with one another. The disclosed methods, systems, and devices are not limited to any specific aspect, feature, or combination thereof, nor do the disclosed methods, systems, and apparatus require that any one or more specific advantages be present or problems be solved. Further, the techniques, methods, operations, steps, etc. described or suggested herein can be performed on a living animal or on a nonliving simulation. Living animals include human patients, veterinary patients, and research animals. Nonliving simulations include methods performed on a cadaver, cadaver heart, simulator (e.g. with the body parts, tissue, etc. being simulated), anthropomorphic phantom, etc. In some instances, a non-living simulation is utilized for training purposes. In some instances, a nonliving tissue wall is used install a cinch ring system, simulating a procedure that can be performed within an animal.

Various examples of systems and examples of prosthetic implants are disclosed herein, and any combination of these options can be made unless specifically excluded. For example, various descriptions of cinching systems can be delivered and implanted by any appropriate method, even if a specific combination is not explicitly described. Likewise, the different constructions and features of devices and systems can be mixed and matched, such as by combining any implant device type/feature, attachment type/feature, site of repair, etc., even if not explicitly disclosed. In short, individual components of the disclosed systems can be combined unless mutually exclusive or physically impossible.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods, systems, and apparatus can be used in conjunction with other systems, methods, and apparatus.

### Cinch Ring Systems for Shunting and Plugging

Several examples are directed to the use of a cinching system for shunting or plugging within a living animal or a non-living simulation. In many of these examples, a cinching system is used for shunting or plugging within the cardiovascular system of a living animal or within a non-living simulation of the cardiovascular system. Generally, a cinching system comprises a set of two or more rings and at least one cord. Although the various depictions and descriptions of the cinching system are provided as utilizing two or more rings, any shape can be utilized. A cinching system can further comprise various components that may help delivery via a transcatheter.

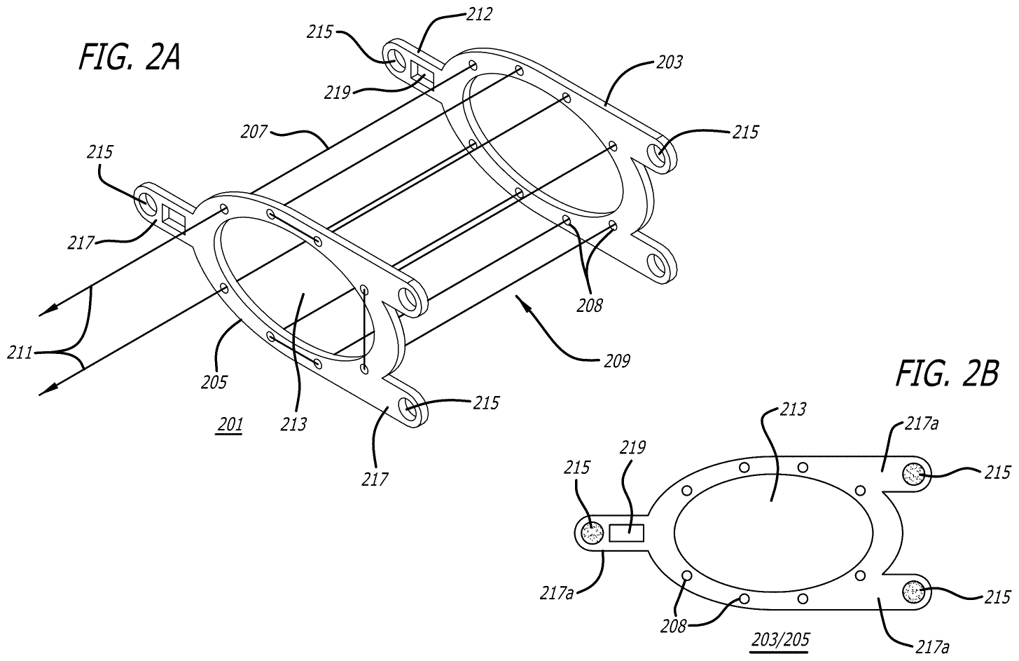

Provided in FIG. 2A is an example of a cinching system 201 that may be utilized for providing a shunt within the cardiovascular system of a living animal or within a non-living simulation of the cardiovascular system. FIG. 2B provides an example of a ring (203 or 205) for use within cinching system 201. As shown, cinching system 201 includes a distal ring 203 and a proximal ring 205 and a cord 207 that is stitched along and between the two rings, through a plurality of stitch holes 208. Alternatively, a cord, or a set of cords, can be looped along the rings and or secured or connected in any fashion that allows for a cinching mechanism to occur.

When installed, each of the two rings are in parallel planes (or near parallel planes) such that proximal face of distal ring 203 faces the distal face of proximal ring 205. The stitching of cord 207 connects the two rings, but can be initially provided loosely such that there is a space 204 between the planes of the rings. Space 204 can vary, and the amount of space required between the two rings will depend on the application and procedure for installing the cinching system.

As shown, a single cord (cord 207) is utilized in the cinching system, with the two ends 211 of the cord being near to one another on the proximal side of proximal ring 205, which allows both ends to be pulled concurrently. Pulling cord ends 211 concurrently in the proximal direction tightens cord 207 and pulls distal ring 203 and proximal ring 205 together, reducing space 204.

Distal ring 203 and proximal ring 205 can each have a central aperture 213 and ring shape that are similarly sized, which may be useful for implanting on a tissue wall (see Delivery systems and modes of delivery for more on implantation). The aperture and ring shape can be any size or shape that allows for shunting on a tissue wall. In some instances, the ring shape can be any size is further capable of being sheathed within a transcatheter for delivery and installation. In some instances, the ring shape is capable of conforming into a deliverable conformation, which can allow it to fit with an outer sheath of a catheter. In some instances, the ring shape is an elongated shape (e.g., ovalular, rectangular) such that it can fold or curve inwards when sheathed within a transcatheter, providing larger ring size with less profile during delivery. In some instances, the central aperture of the ring has an area that is greater than the cross-sectional area of the transcatheter.

Distal ring 203 and proximal ring 205 can each contain a number of radiopaque markers 215, strategically placed such that the orientation of the ring can be visualized via radiographic techniques during implantation. In some instances, and as shown here, three radiopaque markers 215 are provided as apertures with a marker band. In some instances, three radiopaque markers 215 are provided in an orientation relative to one another such that the opaque markers lack mirrored symmetry along one central axis of the ring. As shown in the example of FIG. 2B, the opaque markers are in a triangular orientation and lacking mirrored symmetry along the shorter central axis of the elongated ovular ring. Furthermore, the radiopaque markers 215 of the proximal ring 205 are in an orientation that mirrors the radiopaque markers 215 of the distal ring 203 such that they can be aligned when the proximal ring and the distal ring are provided in parallel planes.

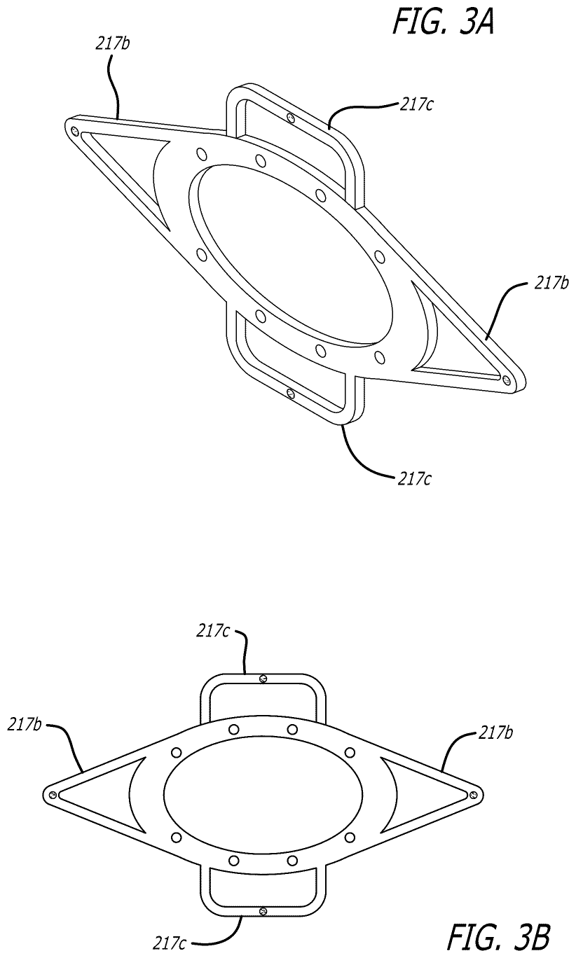

Distal ring 203 and proximal ring 205 can each contain a number of extended protrusions 217, which can increase the profile size of each ring when installed. Extended protrusions can be arms, struts or any other extremity that protrudes outwardly from the ring shape. FIGS. 3A and 3B provide examples of extended protrusions 217 that increase profile size when installed. In some instances, the extended protrusions have a low profile within a transcatheter. Accordingly, in some instances, extended protrusions are longer in one axial direction. In some instances when an elongated shape of ring is utilized, the longer extended protrusions are elongated in the same axial direction as the elongated shape of the ring. For instance, extended protrusions 217a of FIG. 2B and extended protrusions 217b of FIG. 3B are in the same axial direction of the longer central axis of the elongated ovular ring. Further, in some instances, extended protrusions are capable of being folded or curved inward within a transcatheter to minimize profile. For instance, extend protrusions 217c of FIG. 3B are capable of being folded or curved toward the longer central axis of the elongated ovular ring.

Distal ring 203 and proximal ring 205 can each further contain a number of windows, such as window 219 in FIGS. 2A and 2B. Windows can reduce the amount of material of a ring and may provide attachment points for use within a transcatheter delivery system.

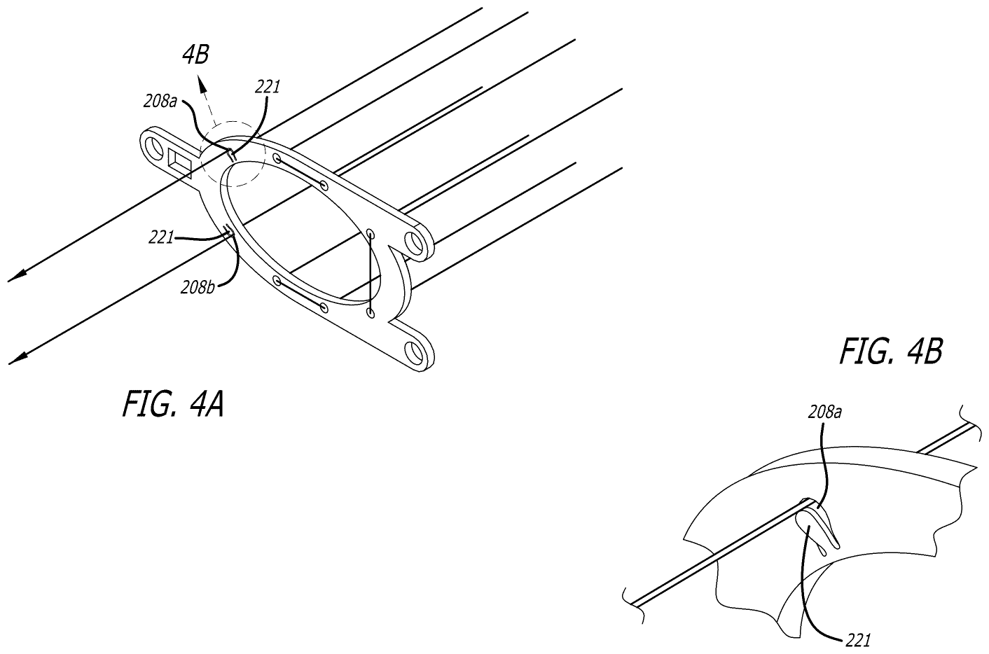

The rings of a cinching system can further incorporate a cord grip on one or more stitch holes. A cord grip provides a means for securing the cord such that the cord can proceeds through the stitch hole in one direction as it tightens and prevents loosening and backtracking of the cord. As shown in FIGS. 4A and 4B, proximal ring 205 incorporates a cord grip 221 on each of the two exit stitch holes 208a and 208b. Cord grips 221 are each a flexible yet rigid tongue that pinches cord 207 against the edge of exit stitch hole, allowing the cord to proceed in to be pulled in the proximal direction and preventing the cord from sliding back in the distal direction. Although a pinching tongue is shown, a cord grip can utilize any mechanism that allows the cord to proceed in to be pulled in the proximal direction and preventing the cord from sliding back in the distal direction. A cord grip can further contain a release mechanism, loosening the cord and allowing the cord to slide back in the distal direction, which may be useful during installation if (for example) the cinching system needs to be moved or resituated.

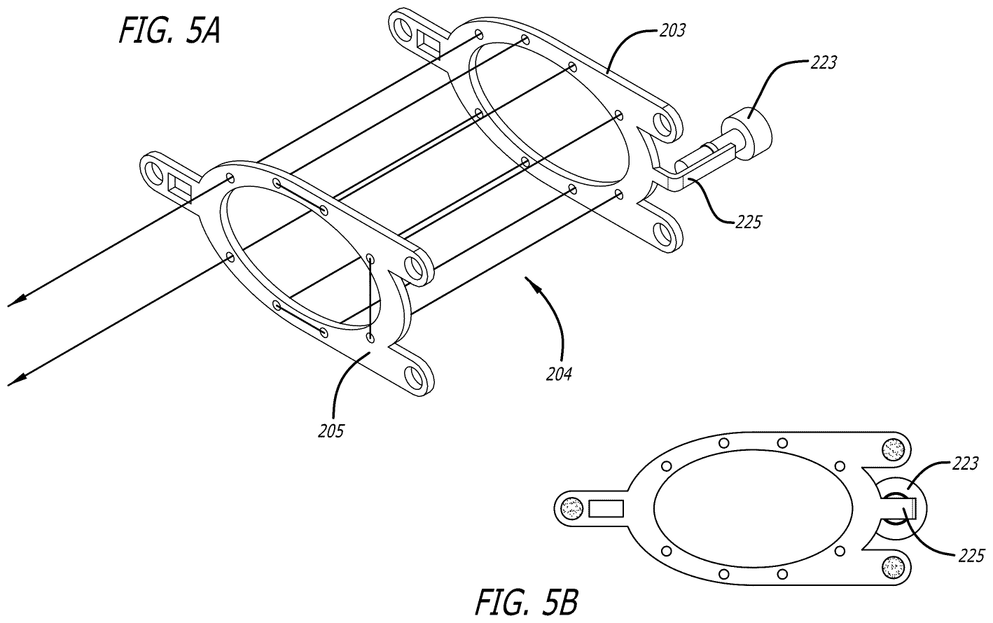

One or more rings of a cinching system can further incorporate a sensor. Generally, a sensor can be secured to a protruding arm that extends from a ring, extending in a direction away from the central space between the rings. FIGS. 5A and 5B provide an example in which distal ring 203 incorporates a sensor 223 that is secured to a protruding arm 225 that extends in the distal direction away from space 204. Although sensor 223 is shown to be extending from distal ring 203, a sensor can alternatively or additionally be extended from proximal ring 205 in a proximal direction away from space 204. Any type of sensor can be attached to a cinching ring. In some instances, the sensor is a telemetric sensor. In various instances, the sensor is a pressure sensor, a temperature sensor, a flow rate sensor, oxygen saturation sensor, loop recorder (cardiac monitor), any other hemodynamic sensor, or any combination of sensors.

The rings of a cinching system can each be composed of any biocompatible material. In some instances, the material is malleable such that it can fold or curve within a catheter for delivery. In some instances, the material is a biocompatible metal or metal alloy, which include (but are not limited to) nitinol, stainless steel, cobalt-chromium alloys, titanium, and titanium alloys. In some instances, the material is a biocompatible polymer, which include (but are not limited to) nylon, poly (lactic-co-glycolic) acid (PLGA), polyvinylchloride (PVC), polyethylene (PE), polypropylene (PP), polytetrafluoroethylene (PTFE), polyurethane (PU), polyethyleneterephthalate (PET), polyethersulfone (PES), polyglycolic acid (PGA), polylactic acid (PLA), poly-D-lactide (PDLA), poly-4-hydroxybutyrate (P4HB), polyether ether ketone (PEEK), and polycaprolactone (PCL).

The one or more cords of a cinching system can each be composed of any biocompatible material capable of being provided as a wire, a monofilament, twine, a woven cord, or a micro-cable. In some instances, the material is providing flexibility but maintain high tension strength such that it can be tightened during cinching. In some instances, the material is a biocompatible metal wire or metal alloy wire, which include (but are not limited to) nitinol, stainless steel, cobalt-chromium alloys, titanium, and titanium alloys. In some instances, the material is a biocompatible polymer monofilament or twine, which include (but are not limited to) nylon, poly (lactic-co-glycolic) acid (PLGA), polyvinylchloride (PVC), polyethylene (PE), polypropylene (PP), polytetrafluoroethylene (PTFE), polyurethane (PU), polyethyleneterephthalate (PET), polyethersulfone (PES), polyglycolic acid (PGA), polylactic acid (PLA), poly-D-lactide (PDLA), poly-4-hydroxybutyrate (P4HB), polyether ether ketone (PEEK), and polycaprolactone (PCL). In some instances, the material is a biocompatible surgical grade natural monofilament or twine, which include surgical grade cotton and surgical grade silk.

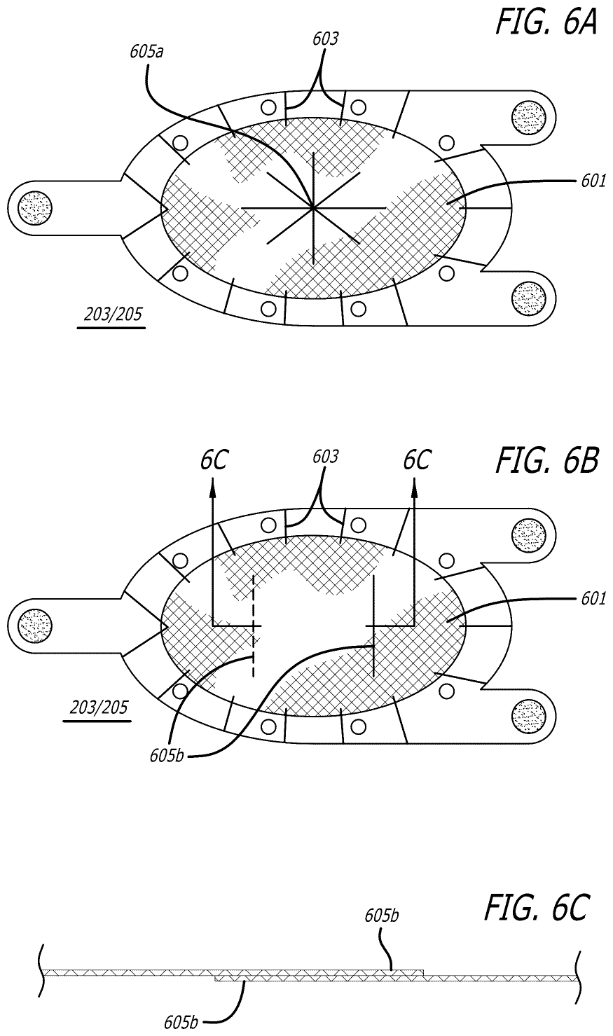

Provided in FIGS. 6A, 6B, and 6C are examples of a ring (203 or 205) for use within a cinching system that may be utilized for providing a plug within the cardiovascular system of a living animal or within a non-living simulation of the cardiovascular system. A cinching system for plugging can incorporate any and/or all of the features and components as described and/or shown of the cinching systems of FIGS. 2A to 5B and further incorporates a cover 601 spanning the central aperture of at least one ring of the system. In some instances, a cover spans the central aperture of the distal ring only. In some instances, a cover spans the central aperture of the proximal ring only. In some instances, a cover spans the central aperture of each ring of the distal and proximal rings.

A cover spanning the central aperture of a ring can be secured to the ring by any mechanism. In some instances, sutures are utilized to stitch the cover onto the ring. In some instances, a biocompatible adhesive is utilized to secure the cover to the ring. In some instances, the cover is crimped and/or pinched onto the ring. In some instances, a cover fully or partially encapsulates the ring like a sleeve. In the examples of FIGS. 6A and 6B, sutures 603 are utilized to secure the cover to the ring.

In several instances, a cover spanning a ring incorporates a through hole or slit, which can assist in delivery and installation of the cinching system (see Delivery system and modes of delivery). In many instances, the through hole or slit is large enough for an inner lumen of a delivery system to pass through. As shown in the example with in FIG. 6A, cover 601 contains a through hole 605a that is asterisk-shaped, which can allow an inner lumen of a delivery system to pass through and once installed, asterisk-shaped through hole 605a can close up and mitigate passage of fluid (e.g., blood). The example provided in FIGS. 6B and 6C provides overlapping slits 605b, which can allow an internal lumen of a delivery system to pass through and once installed, overlapping slits 605b can close up and mitigate passage of fluid (e.g., blood). In various instances, including the examples of FIGS. 6A to 6C, once the cinching system is installed, the through hole or slit can be sealed. In some instances, stitching or a biocompatible adhesive is utilized to securely close the through hole or slit. In some instances, a cinching mechanism is utilized to securely close the through hole or slit. In instances utilizing a cinching mechanism to securely close the through hole or slit, the cinching mechanism can utilize the same cord(s) as the cinching mechanism utilized to bring the two rings together or its own unique cord(s).

In accordance with several instances, a cinching system is delivered to and installed within the cardiovascular system of a living animal or within a non-living simulation of the cardiovascular system. In many instances, a cinching system is delivered to and installed at a location that requires a shunt. Generally, when installing a shunt, a cinching system is delivered to a tissue wall that separates two cavities filled with fluid (e.g., blood), such as (for example) the chambers the heart and lumens of the vasculature. In particular instances, a shunt is delivered to the septum separating the left atrium and right atrium or to the tissue wall separating the left atrium and the coronary sinus, which can relieve pressure in the left atrium and could treat patients suffering from heart failure. In many instances, a cinching system that incorporates a cover spanning the central aperture of a ring is delivered to and installed at a location that requires a plug. Generally, when installing a plug, a cinching system is delivered to an aperture that results in undesired fluid flow or leakage, such as an aneurism or fistula. In particular instances, a plug is delivered to a coronary artery aneurysm (CAA) or a coronary artery fistula (CAF) to treat these conditions.

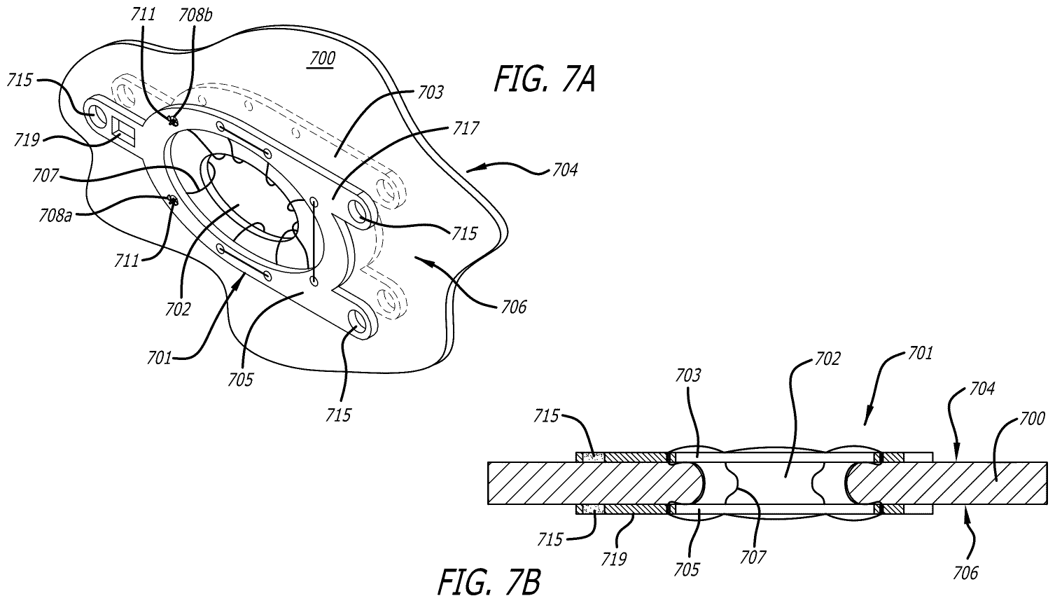

Provided in FIGS. 7A and 7B is an example of a cinching system 701 installed upon a tissue wall 700 for shunting fluid therethrough. A puncture hole 702 has been punctured through the tissue prior to installation of cinching system 701. Cinching system 701 incorporates a distal ring 703 and proximal ring 705 and a cord 707 that is stitched along and between the two rings, through a plurality of stitch holes 708. As shown in the installed position, cord 707 is tightened such that distal ring 703 is adjacent to and in contact with the distal wall face 704 of tissue wall 700 and proximal ring 705 is adjacent to and in contact with the proximal wall face 706 of the tissue. Distal ring 703 and proximal ring 705 are aligned on their respective side of tissue wall 700, forming a mirror image across the tissue (or near mirror image as dependent on local tissue architecture). The alignment of the rings can be achieved utilizing the radiopaque markers 715 during the process of installation. Distal ring 703 and proximal ring 705 further encircle puncture hole 702 with cord 707 traversing the puncture hole multiple times as dependent on the number of stitch holes 708. The traversing of cord 707 can help maintain and stabilize puncture hole 702 in tissue wall 700.

As shown, a single cord (cord 707) is utilized in the cinching system, with the two ends 711 of the cord being near to one another on the proximal side of proximal ring 705, which allows both ends to be pulled concurrently. Cord ends 711 can be secured and/or tied off at the two exit stitch holes 708a and 708b, which can be done utilizing (for example) a cord grip and/or tied knots. Alternatively, multiple cords can be utilized, and each the cord ends of each cord can be secured and/or tied off as described.

Distal ring 703 and proximal ring 705 can each contain a number of extended protrusions 717, which can increase the profile size of each ring installed against the tissue wall 700. Extended protrusions can be arms, struts or any other extremity that protrudes outwardly from the ring shape. Distal ring 703 and proximal ring 705 can each further contain a number of windows, such as window 719.

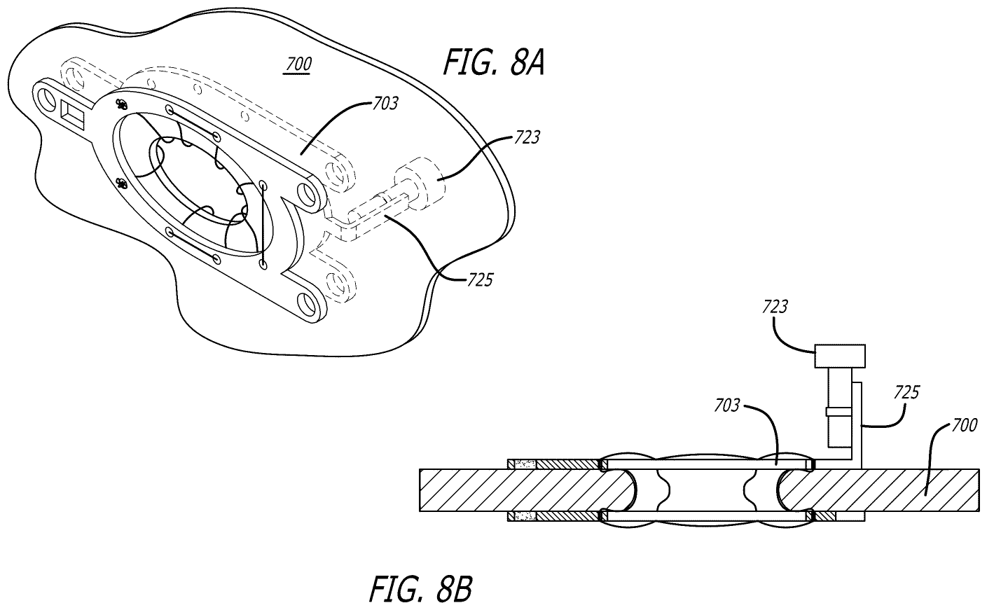

FIGS. 8A and 8B provide an example in which distal ring 703 incorporates a sensor 723 that is secured to a protruding arm 725 that extends in the distal direction away from tissue wall 700. Although sensor 723 is shown to be extending from distal ring 703, a sensor can alternatively or additionally be extended from proximal ring 705 in a proximal direction away from tissue wall 700. Any type of sensor can be attached to a cinching ring. In some instances, the sensor is a telemetric sensor. In various instances, the sensor is a pressure sensor, a temperature sensor, a flow rate sensor, oxygen saturation sensor, loop recorder (cardiac monitor), any other hemodynamic sensor, or any combination of sensors. For instance, when utilized for treating conditions associated with high blood pressure such as heart failure, a pressure sensor can be utilized to monitor the health of the individual and the effectiveness of the shunt.

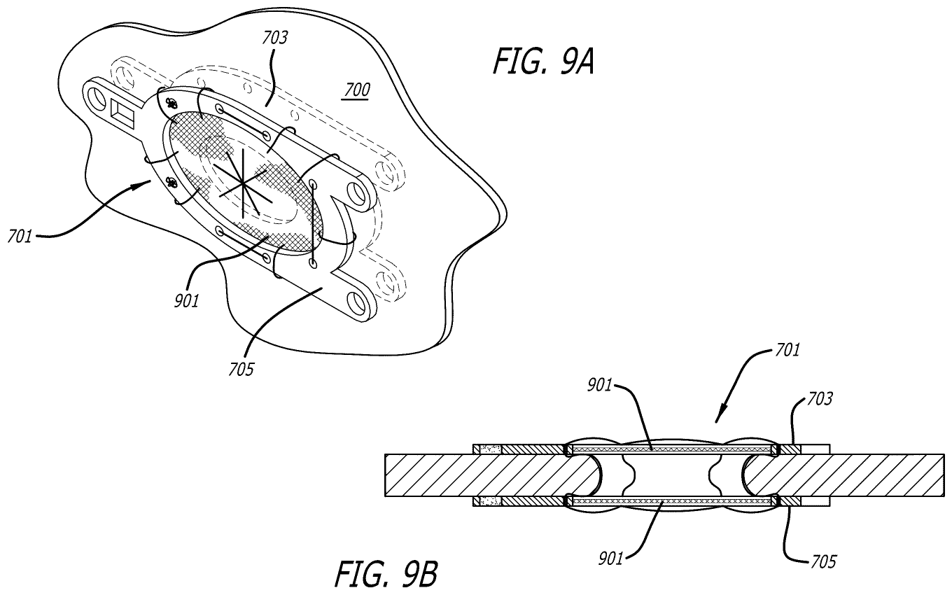

Provided in FIGS. 9A and 9B are examples of a cinching system 701 with a cover 901 installed over an aperture in tissue wall 700, which can be utilized to plug an aneurysm, fistula, or other aperture. As shown, the cinching system 701 for plugging can be installed with any and/or all of the features and components as described and/or shown of the cinching systems of FIGS. 8A to 8B and further incorporates a cover 901 spanning the central aperture of each distal ring 703 and proximal ring 705. Although FIGS. 9A and 9B depict a cover spanning the apertures of each the distal ring and the proximal ring, a cover can span only the distal ring or the proximal ring to plug an aperture.

### Cinch Ring Systems for Device Securement

Several examples are directed to the use of a cinching system for installing a device (e.g., sensor) within a living animal or a non-living simulation. In many of these examples, a cinching system is used for securing a device on a tissue wall or membrane within the cardiovascular system of a living animal or within a non-living simulation of the cardiovascular system. Generally, a cinching system comprises a set of two or more rings, at least one cord, and a device to be secured. Although the various depictions and descriptions of the cinching system are provided as utilizing two or more rings, any shape can be utilized. Furthermore, the examples and drawings depict a sensor as the device to be secured, but any device that can be attached to a cinching system and installed into a tissue wall or membrane can be utilized. A cinching system can further comprise various components that may help delivery via a transcatheter.

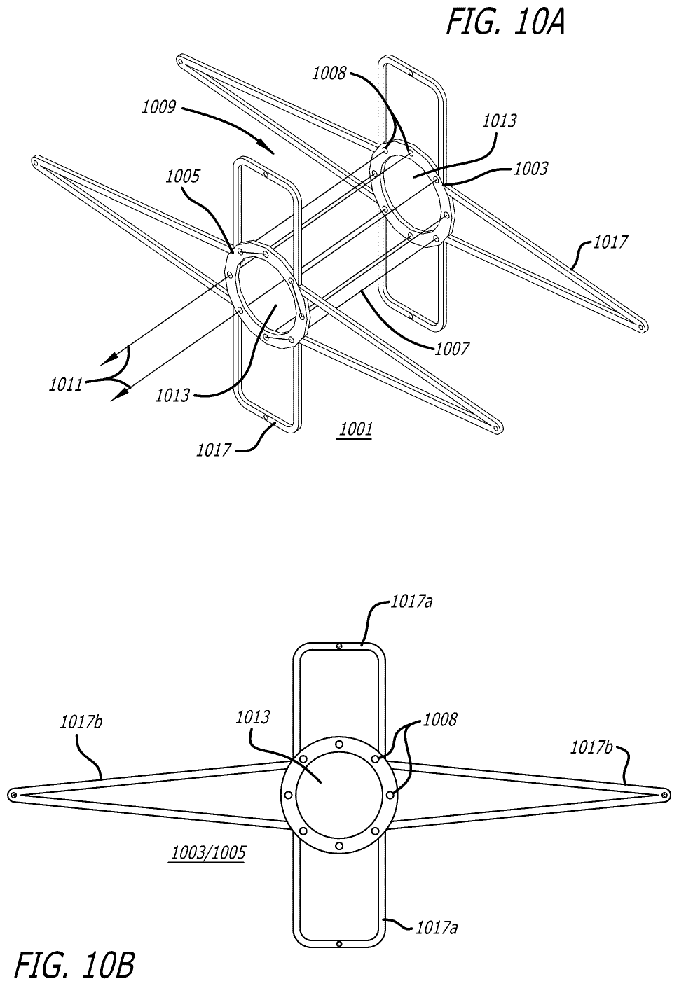

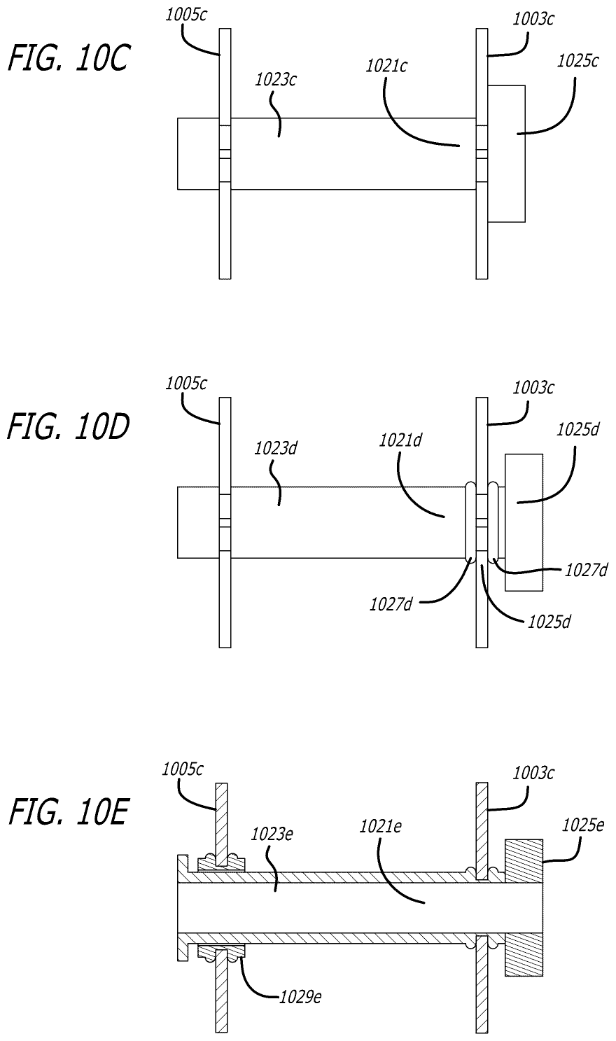

Provided in FIG. 10A is an example of a cinching system 1001 that may be utilized for securing a device within the cardiovascular system of a living animal or within a non-living simulation of the cardiovascular system. FIG. 10B provides an example of a ring (1003 or 1005) for use within cinching system 1001. As shown, cinching system 1001 includes a distal ring 1003 and a proximal ring 1005 and a cord 1007 that is stitched along and between the two rings, through a plurality of stitch holes 1008. Alternatively, a cord, or a set of cords, can be looped along the rings and or secured or connected in any fashion that allows for a cinching mechanism to occur.

When installed, each of the two rings are in parallel planes (or near parallel planes) such that proximal face of distal ring 1003 faces the distal face of proximal ring 1005. The stitching of cord 1007 connects the two rings, but can be initially provided loosely such that there is a space 1009 between the planes of the rings. Space 1009 can vary, and the amount of space required between the two rings will depend on the application and procedure for installing the cinching system.

As shown, a single cord (cord 1007) is utilized in the cinching system, with the two ends 1011 of the cord being near to one another on the proximal side of proximal ring 1005, which allows both ends to be pulled concurrently. Pulling cord ends 1011 concurrently in the proximal direction tightens cord 1007 and pulls distal ring 1003 and proximal ring 1005 together, reducing space 1009.

Distal ring 1003 and proximal ring 1005 can each have a central aperture 1013 and ring shape that are similarly sized, which may be useful for implanting on a tissue wall and securing a device (see Delivery systems and modes of delivery for more on implantation). The aperture and ring shape can be any size or shape, but should be shaped concordantly with a device body. Devices can be attached within or secured to the central aperture to the proximal, the distal, or both rings, while still allowing the rings to be cinched together.

Provided in FIGS. 10C, 10D, and 10E are examples of cinch ring systems having an associated sensor 1021 that is secured to cinch ring system 1001. Any type of sensor can be associated to a cinch ring system. In some instances, the sensor is a telemetric sensor. In various instances, the sensor is a pressure sensor, a temperature sensor, a flow rate sensor, oxygen saturation sensor, loop recorder (cardiac monitor), any other hemodynamic sensor, or any combination of sensors.

In the example of FIG. 10C, sensor body 1023c and/or the sensor head 1025c of sensor 1021c is attached to distal ring 1003c. The attachment of 1021c to distal ring 1003c can be any attachment that is secure, such as (for example) an adhesive, a staple, a suture, a welding, a rivet, a screw, or any other fastening mechanism. Distal ring 1003c can be fixed to sensor 1021c such that the distal ring holds the sensor in a fixed position relative to the installation site. Proximal ring 1005c is not attached to sensor 1021c but is slidable along sensor body 1023c such that a cinching cord (or set of cinching cords) can be utilized to install and secure the cinch ring system and sensor at the installation site. Although distal ring 1003c is described as being attached to sensor 1021c, it is to be understood that proximal ring 1005c can be attached to and/or fixed to the sensor and the distal ring is not attached but is slidable along sensor body 1023c. It is to be further understood that both distal ring 1003c and proximal ring 1005c can both not be attached to sensor 1021c and slidable along sensor body 1023c such that the two rings can slide towards one another when cinched. In instances utilizing slidable proximal and distal rings, a ridge extending outwardly from sensor body 1023c and kept in between the two rings can be utilized to hold the sensor in place when installed and secured.

In the example of FIG. 10D, sensor body 1023d of sensor 1021d includes a groove 1025d and a set of ridges 1027d that secure and hold distal ring 1003d in place. Groove 1025d and ridges 1027d can hold distal ring 1003d in relative position to sensor 1021d such that the distal ring holds the sensor in a fixed position relative to the installation site. Proximal ring 1005d is not attached to sensor 1021d but is slidable along sensor body 1023d such that a cinching cord (or set of cinching cords) can be utilized to install and secure the cinch ring system and sensor at the installation site. Although distal ring 1003d is described as being held in fixed position relative to sensor 1021d, it is to be understood that proximal ring 1005c can be held in fixed position relative to the sensor and the distal ring is not attached but is slidable along sensor body 1023d.

In the example of FIG. 10E, sensor body 1023e of sensor 1021e holds distal ring 1003c in place and includes an outer sheath 1029e that holds proximal ring 1005e in place. Outer sheath 1029e is slidable along sensor body 1023e such that proximal ring 1005e can slide along the senor body. A cinching cord (or set of cinching cords) can be utilized to slide outer sheath 1029e and proximal ring 1005e to install and secure the cinch ring system and sensor at the installation site. Although distal ring 1003d is described as being held in fixed position relative to sensor 1021d and proximal ring 1005e is held in place by a slidable outer sheath 1029e, it is to be understood that the proximal ring can be held in fixed position relative to the sensor and the distal ring is held in place by an outer sheath that is slidable along sensor body 1023e. It is to be further understood that both distal ring 1003e and proximal ring 1005e can be held in place by their own respective outer sheath that slidable along sensor body 1023e, such that the two rings can slide towards one another when cinched. In instances utilizing proximal and distal rings that are each attached to their own respective slidable outer sheath, a ridge extending outwardly from sensor body 1023c and kept in between the two rings can be utilized to hold the sensor in place when installed and secured.

Distal ring 1003 and proximal ring 1005 can each contain a number of extended protrusions 1017, which can increase the profile size of each ring when installed. Extended protrusions can be arms, struts or any other extremity that protrudes outwardly from the ring shape. Extended protrusions can have any shape, such as a singular extended strut, a triangular extension, a rectangular extension, etc. When utilizing a cinch ring system for installation of small device (e.g., sensor), longer protrusions may be desired. In some instances, one or more protrusions have an extended length between 1X and 5X of the internal diameters. In some instances, protrusions have an extended length at least 1X, an extended length of at least 2X, an extended length of at least 3X, an extended length of at least 4X, or an extended length of at least 5X of the internal diameter of the ring. Protrusion shape and length may depend, in part, on the installation site anatomy, as it may be desired and/or necessary to conform to the shape of the local anatomy. As shown in the example of FIG. 10B, protrusions 1017a are rectangular in shape and extend outwardly about 1X of the internal diameter of the ring and protrusion 1017b are triangular in shape and extend outwards about 3x of the internal diameter of the ring.

In some instances, the ring shape and protrusions can be any size or shape that is capable of being sheathed within a transcatheter for delivery and installation. In some instances, the shape and size of the ring and/or protrusions are capable of conforming into a deliverable conformation, which can allow it to fit within an outer sheath of a catheter. In some instances, the ring shape is round and has a total area less than the cross-sectional area of the outer sheath such that it can fit within a transcatheter in a position parallel to a cross-section of the outer sheath. In some instances, the protrusions extending from the ring are capable of bending or folding in a direction that is perpendicular (or substantially perpendicular) of the face of the ring to enable the ring and the protrusions to fit with the outer sheath of a transcatheter device.

The rings of a cinch ring system can each be composed of any biocompatible material. In some instances, the material is malleable such that it can fold or curve within a catheter for delivery. In some instances, the material is a biocompatible metal or metal alloy, which include (but are not limited to) nitinol, stainless steel, cobalt-chromium alloys, titanium, and titanium alloys. In some instances, the material is a biocompatible polymer, which include (but are not limited to) nylon, poly (lactic-co-glycolic) acid (PLGA), polyvinylchloride (PVC), polyethylene (PE), polypropylene (PP), polytetrafluoroethylene (PTFE), polyurethane (PU), polyethyleneterephthalate (PET), polyethersulfone (PES), polyglycolic acid (PGA), polylactic acid (PLA), poly-D-lactide (PDLA), poly-4-hydroxybutyrate (P4HB), polyether ether ketone (PEEK), and polycaprolactone (PCL).

The one or more cords of a cinch ring system can each be composed of any biocompatible material capable of being provided as wire, monofilament, or twine. In some instances, the material is providing flexibility but maintain high tension strength such that it can be tightened during cinching. In some instances, the material is a biocompatible metal wire or metal alloy wire, which include (but are not limited to) nitinol, stainless steel, cobalt-chromium alloys, titanium, and titanium alloys. In some instances, the material is a biocompatible polymer monofilament or twine, which include (but are not limited to) nylon, poly (lactic-co-glycolic) acid (PLGA), polyvinylchloride (PVC), polyethylene (PP), (PE), polypropylene polytetrafluoroethylene (PTFE), polyurethane (PU), polyethyleneterephthalate (PET), polyethersulfone (PES), polyglycolic acid (PGA), polylactic acid (PLA), poly-D-lactide (PDLA), poly-4-hydroxybutyrate (P4HB), polyether ether ketone (PEEK), and polycaprolactone (PCL). In some instances, the material is a biocompatible surgical grade natural monofilament or twine, which include surgical grade cotton and surgical grade silk.

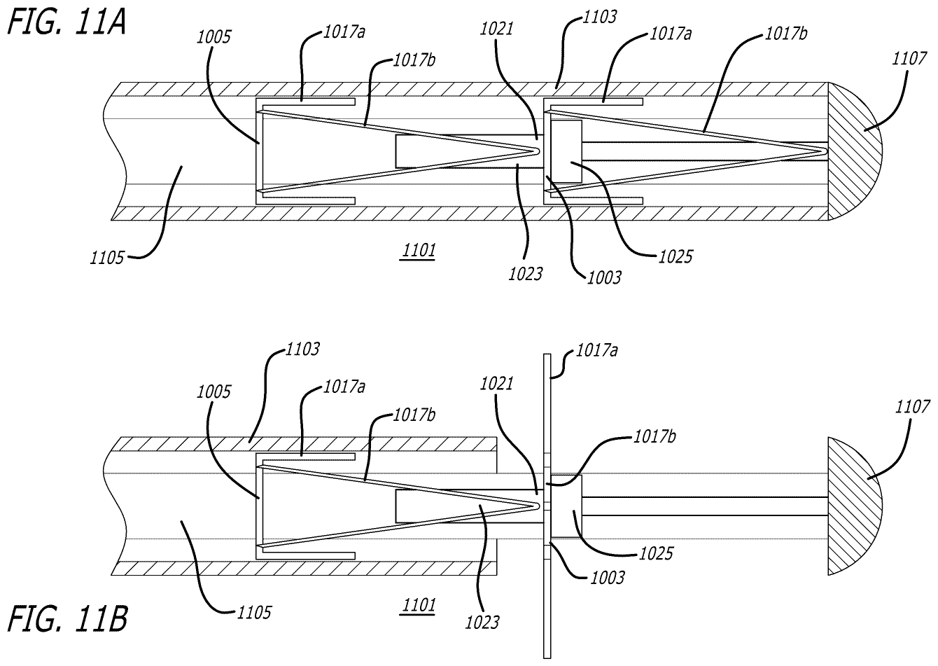

Provided in FIGS. 11A and 11B is an example of the cinch ring system 1001 within a transcatheter delivery system 1101. The transcatheter delivery system comprises an outer sheath 1103, an inner sheath member 1105, and a nose cone 1107. Cinch ring system 1001 includes distal ring 1003 and proximal ring 1005, each kept within outer sheath 1103 and in connection with inner sheath member 1105. The faces of distal ring 1003 and proximal ring 1005 are kept parallel to a cross section of the outer sheath. Protrusions 1017a and 1017b of distal ring 1003 and proximal ring 1005 are folded in a perpendicular position to the faces of the rings, keeping the profile of transcatheter delivery system 1101 minimal. Sensor body 1023 of sensor 1021 is in association with distal ring 1003 and proximal ring 1005, allowing it to be delivered concurrently with cinch ring system 1001. As shown in FIG. 11B, when cinch ring system 1001 is to be installed, distal ring 1003 can be exposed from outer sheath 1103, allowing protrusion 1017 to extend outwardly, such that the cinch ring system can be secured to a tissue wall or membrane. Cinch ring system 1001 is to be installed such that sensor head 1025 is within a cavity, lumen, or other space such that it can record local hemodynamics (e.g., pressure) at the site of installation.

The distal and proximal rings can each contain a number of radiopaque markers that are strategically placed such that the orientation of the ring can be visualized via radiographic techniques during implantation. In addition, the protrusions can be utilized as radiopaque markers to help orient and install the cinch ring system. In some instances, radiopaque markers and/or protrusions are provided in an orientation relative to one another such that the opaque markers and/or protrusions lack mirrored symmetry along one central axis of the ring.

The rings of a cinching system can further incorporate a cord grip on one or more stitch holes. A cord grip provides a means for securing the cord such that the cord can proceeds through the stitch hole in one direction as it tightens and prevents loosening and backtracking of the cord. A cord grip can utilize any mechanism that allows the cord to proceed in to be pulled in the proximal direction and preventing the cord from sliding back in the distal direction. A cord grip can further contain a release mechanism, loosening the cord and allowing the cord to slide back in the distal direction, which may be useful during installation if (for example) the cinching system needs to be moved or resituated.

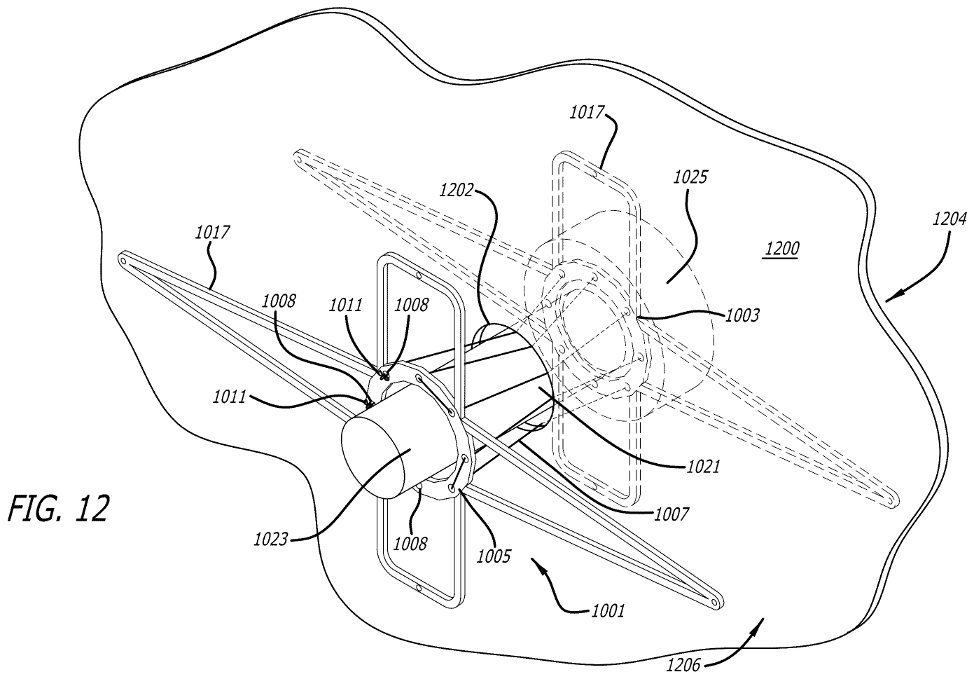

FIG. 12 provides an example of cinch ring system 1001 with an associated sensor 1021 installed at a target site. A puncture hole 1202 has been punctured through the tissue prior to installation of cinching system 1001. Notably, for installation of devices, hole punctures should be kept minimal such that cinch ring system and senor can plug the punctured hole when installed. Cinching system 1001 incorporates a distal ring 1003 and proximal ring 1005 and a cord 1007 that is stitched along and between the two rings, through a plurality of stitch holes 1008. As shown in the installed position, cord 1007 is tightened such that distal ring 1003 is adjacent to and in contact with the distal wall face 1004 of tissue wall 1200 and proximal ring 1005 is adjacent to and in contact with the proximal wall face 1206 of the tissue. Sensor body 1023 traverses through the punctured hole such that sensor head 1025 is exposed on the distal side of tissue wall 1200. It is to be understood that sensor 1021 can be installed such that sensor head 1025 is exposed on the proximal side of the tissue wall, depending on how the sensor is oriented within the transcatheter during delivery. Distal ring 1003 and proximal ring 1005 are aligned on their respective side of tissue wall 1200, forming a mirror image across the tissue (or near mirror image as dependent on local tissue architecture). The alignment of the rings can be achieved utilizing the radiopaque markers and/or extended protrusions during the process of installation. Distal ring 1003 and proximal ring 1005 encircle puncture hole 702 with cord 1007 traversing the puncture hole multiple times as dependent on the number of stitch holes 1008. The cinching of cord 1007 and tightening of distal ring 1003 and proximal ring 1005 on tissue wall 1200 can pinch the tissue together to help create a seal around sensor body 1023 such that fluid (e.g., blood or serum) does not traverse through the puncture hole when installed. Further, attachment of the distal ring 1003 or proximal ring 1005 to sensor body 1023 and/or a ridge extending from the sensor body can also help prevent fluid traversing through the puncture hole.

As shown, a single cord (cord 1007) is utilized in the cinching system, with the two ends 1011 of the cord being near to one another on the proximal side of proximal ring 1005, which allows both ends to be pulled concurrently. Cord ends 1011 can be secured and/or tied off at the two exit stitch holes 1008a and 1008b, which can be done utilizing (for example) a cord grip and/or tied knots. Alternatively, multiple cords can be utilized, and each the cord ends of each cord can be secured and/or tied off as described.

Distal ring 1003 and proximal ring 1005 can each contain a number of extended protrusions 1017, which can increase the profile size of each ring installed against the tissue wall 1200. Extended protrusions can be arms, struts or any other extremity that protrudes outwardly from the ring shape.

### Delivery Systems and Modes of Delivery

Several examples are directed towards delivery systems incorporating cinching systems and methods to deliver and install cinching systems. Generally, a cinching system can be installed in the body via any surgical procedure that can reach the target site of implantation. Methods of delivery include (but not limited to) open heart surgery and transcatheter delivery. When a transcatheter delivery system is used, any appropriate approach may be utilized to reach the site of deployment, including (but not limited to) a transfemoral, subclavian, transapical, or transaortic approach. Because transcatheter delivery approach utilizes a catheter to travel through the circulatory system to the site of installation, it is desirable to keep the profile of the cinching system within the catheter small such that it can easily traverse the circulatory system, mitigating injury and pain.

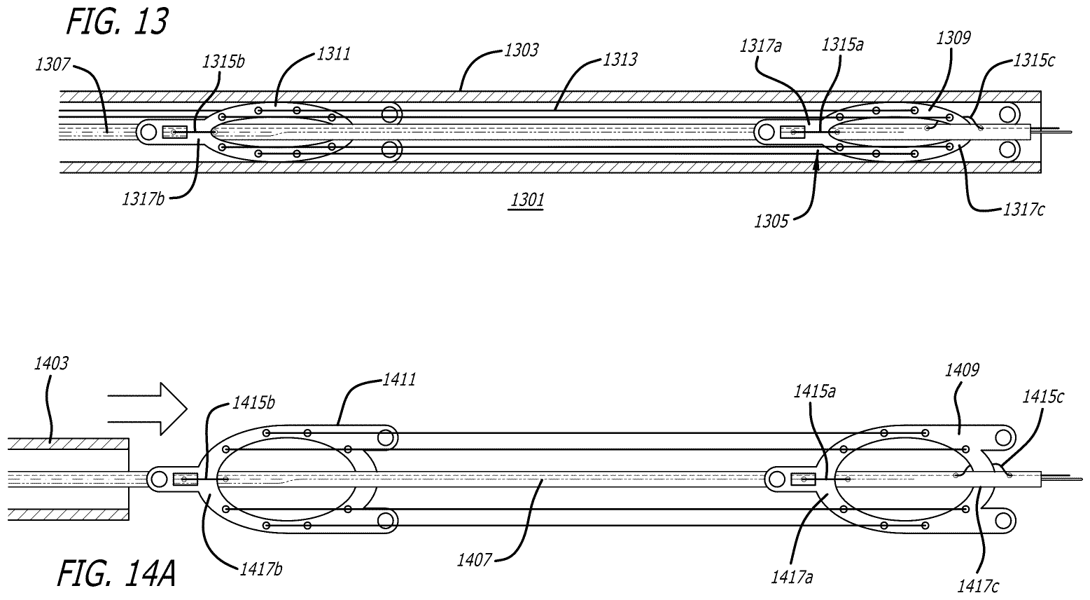

Many examples are directed to methods of sheathing a cinching system within a catheter and preparing delivery systems. In several instances, a delivery system comprises a delivery handle and loaded catheter comprising an outer sheath, an inner sheath, a cinch ring system, and a release wire mechanism for deploying the cinch ring system. Provided in FIG. 13 is an example a transcatheter 1301 for deploying a cinch ring system. The transcatheter comprises an outer sheath 1303 housing a cinch ring system 1305 and an inner sheath 1307. Outer sheath 1303 and inner sheath 1307 are each independently capable of moving in a proximal direction or in a distal direction. Inner sheath 1307 traverses through the central aperture of a distal ring 1309 and the central aperture of a proximal ring 1311. In this example, distal ring 1309 and proximal ring 1311 are curved inward (like a taco shell) to fit within outer sheath 1303 and keep the profile of the transcatheter minimal. Further, distal ring 1309 is kept distant from proximal ring 1311 such to space out the components with greater profile to facilitate traversing through the vasculature system. Cinch ring system 1305 is loaded within the catheter with a cord 1313 linking distal ring 1309 and proximal ring 1311 and ready to be cinched for quick installment once deployed. Three release wires 1315 secure distal ring 1309 and proximal ring 1311 to inner sheath 1307. Release wire 1315a secures a proximal portion 1317a of distal ring 1309. Release wire 1315b secures proximal portion 1317b of proximal ring 1311. And release wire 1315c secures distal portion 1317c of distal ring 1309. (Note that the proximal and distal portions of the rings described here are in reference to their position within the catheter and not as installed.) Although the example of FIG. 13 illustrates a release wire mechanism of having three release wires securing the rings as shown and described, various other release mechanisms for deploying a cinch ring system can be utilized.

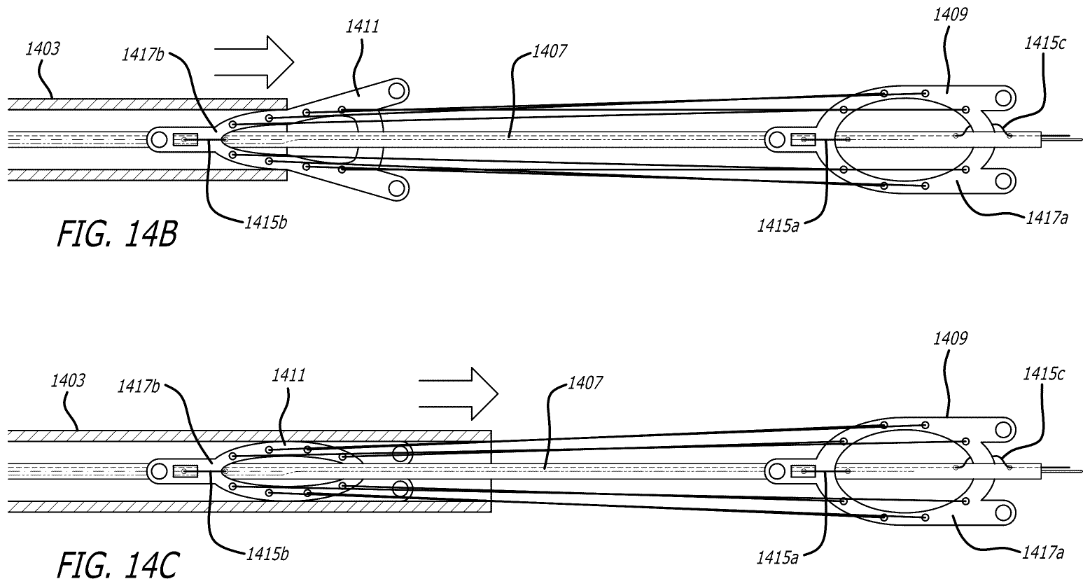

FIGS. 14A to 14C illustrate an exemplary method for sheathing cinch ring system. Prior to sheathing cinch ring system within outer sheath 1403, release wires 1415 secure distal ring 1409 and proximal ring 1411 to inner sheath 1407. Specifically, release wire 1415a secures a proximal portion 1417a of distal ring 1409. Release wire 1415b secures proximal portion 1417b of proximal ring 1411; and release wire 1415c secures distal portion 1417c of distal ring 1409. To sheath the cinch ring system, outer sheath 1403 is pushed in distal direction and/or inner sheath 1407 is pulled in a proximal direction (FIG. 14A). As proximal ring 1411 enters within outer sheath 1403, it curves inward, folding up like a taco shell, such that the profile of the proximal ring is reduced (FIG. 14B). Outer sheath 1403 is continued to be pushed in distal direction and/or inner sheath 1407 I pulled in the proximal direction until the entirety of proximal ring 1411 is sheathed within the outer sheath (FIG. 14C). Distal ring 1409 is sheathed in a similar manner, resulting in a transcatheter ready for deployment as shown in FIG. 13.

Several examples are directed towards methods of delivering and deploying a cinch ring system at the site of installation. In many instances, a transcatheter system is utilized to deliver and deploy the cinch ring system. Generally, a catheter comprising the cinch ring system and release system is delivered to a tissue wall that has been punctured (or containing an aperture to be plugged). The catheter and release system deploy the cinch ring system such that a distal ring is installed on the distal side of the punctured tissue wall and a proximal ring is installed on the proximal side of the wall. The delivery system is capable of tightening a cord that connects the distal and proximal rings to complete installation via a cinching mechanism.

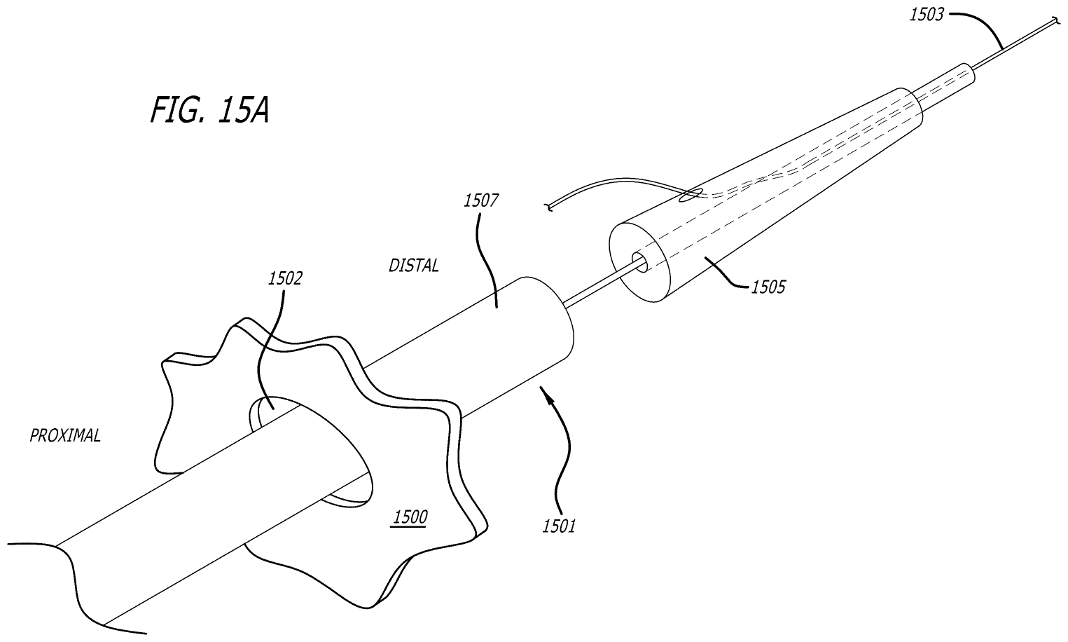

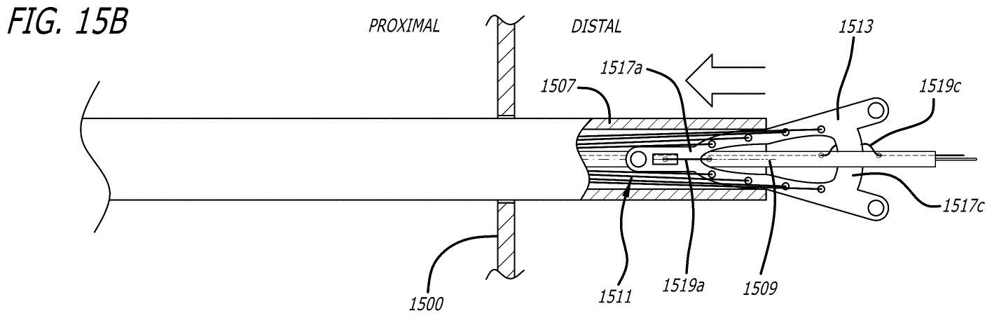

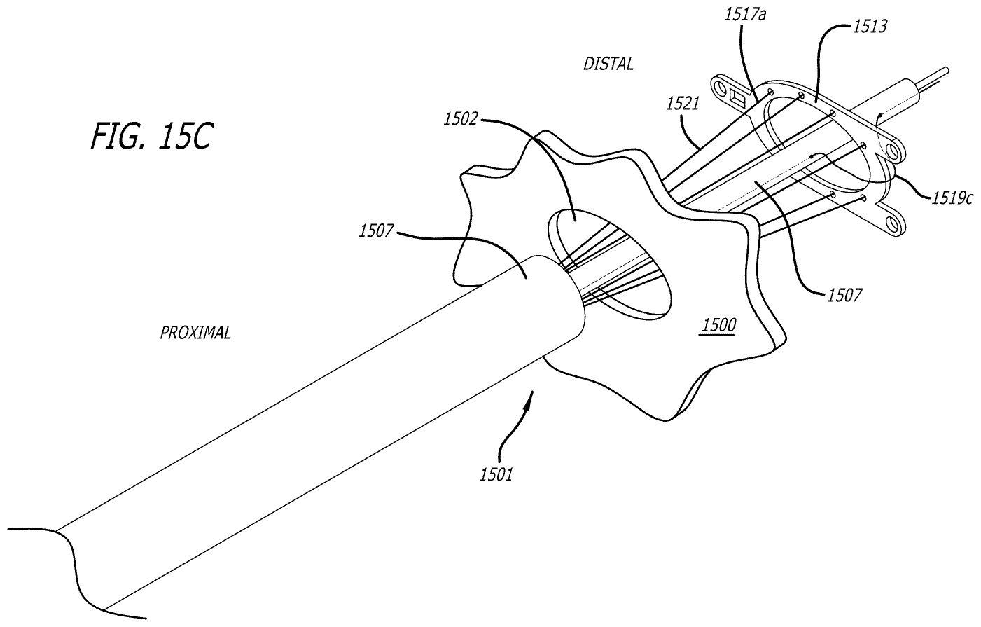

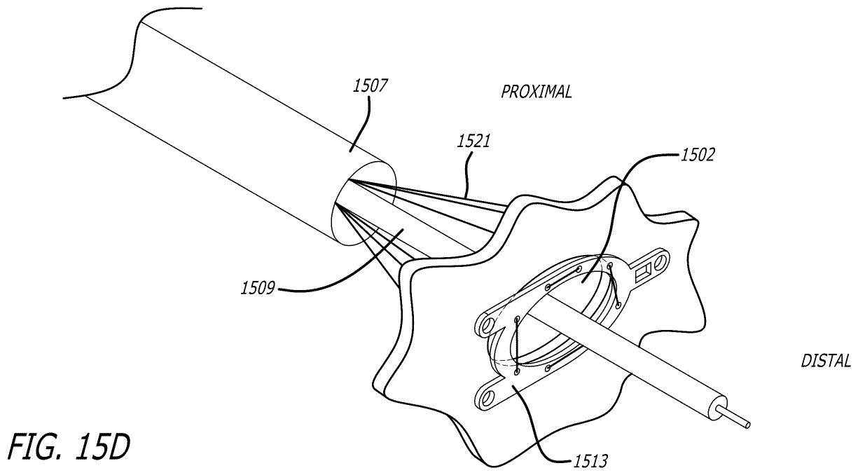

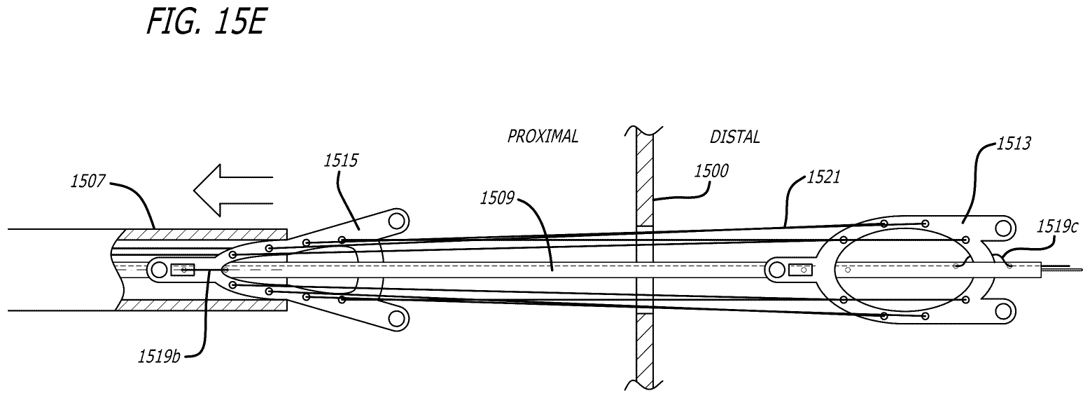

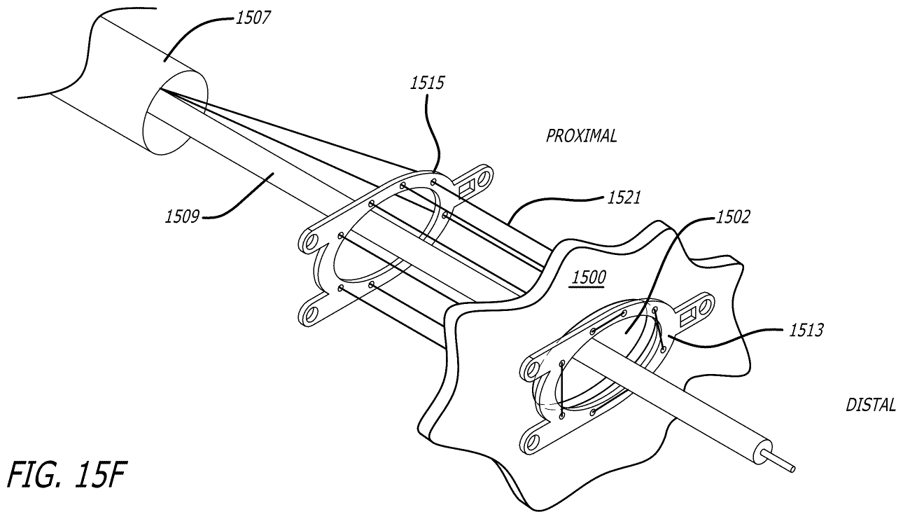

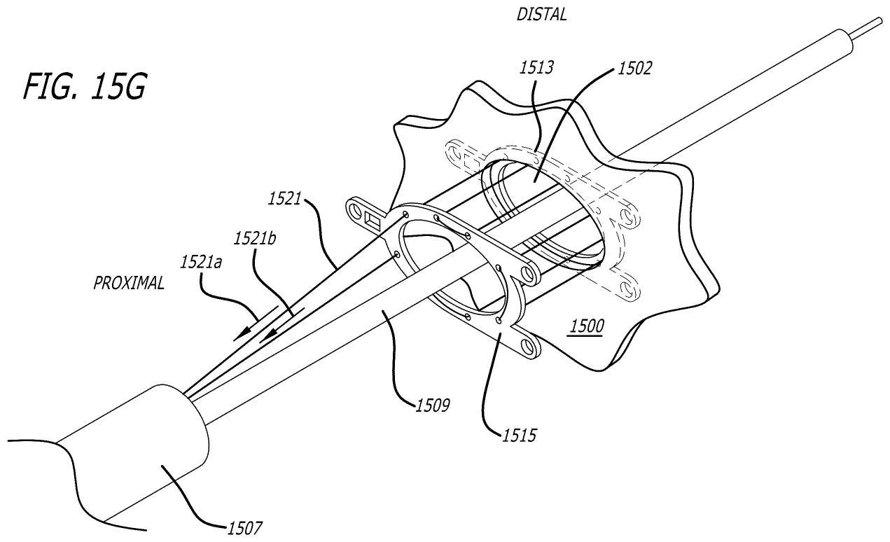

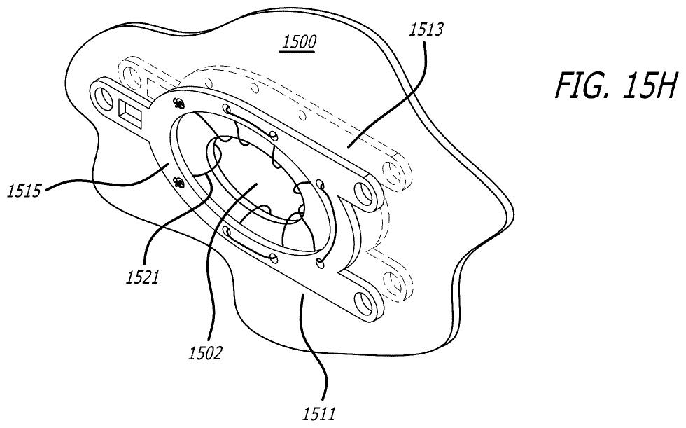

Provided in FIGS. 15A to 15H is an exemplary method of delivering and installing a cinch ring system to a target site via a transcatheter delivery system. For transcatheter delivery, an incision is made at a location with access to the circulatory system, such as (for example) the femoral artery and the transcatheter is inserted and travels through the circulatory system to the site of installation. The precise location of incision and entry can vary, and can depend on the site of installation, health of the patient, and other considerations. Although the example in these figures depict delivery of a cinch ring system for shunting, the general concepts and method of delivery will be similar for the other cinch ring systems (e.g., for plugging, or for device installation) with only minor deviations based on the end goal.

FIG. 15A depicts a tissue wall 1500 with an aperture 1502 within the tissue wall. When providing a shunt, a procedure can be performed to puncture the aperture within the tissue wall. Generally, a transcatheter system delivers a puncturing tool to the target site, punctures an appropriately sized aperture, and removes the punctured tissue debris from the target site. In some instances, the puncture site is dilated, which can be done utilizing an inflatable balloon or a tapered dilator. An appropriately sized aperture depends on the size of shunt to be generated, but will typically be larger than the circumference of the outer sheath of the catheter but smaller than the central aperture of the rings of a cinch ring system. Alternatively, when providing a plug, a puncture procedure is typically not needed, but some excision of tissue and removal may be needed prior to delivery of the cinching ring system.

As shown in FIG. 15A, a transcatheter system 1501 is delivered to tissue wall 1500 with aperture 1502 via a guide wire 1503. A nose cone 1505 is utilized to help the transcatheter system 1501 travel through the circulatory system and through aperture 1502 to the distal side of tissue wall 1500. Nose cone 1505 can be released from an outer sheath 1507, allowing the contents such as the cinching ring system within the outer sheath to be installed.

FIG. 15B depicts the initial exposure of distal ring 1513 of the cinching ring system 1511 in the space distal to tissue wall 1500. Distal ring 1513 can contain two attachment points to inner sheath 1509, each attachment point having its own release wire (1519a and 1519c). Release wire 1519a attaches at a proximal point 1517a on distal ring 1513 and release wire 1519c attaches at a distal point 1517c on distal ring 1513. To expose distal ring 1513, outer sheath 1507 can be slide in the proximal direction and/or inner sheath 1509 can be slide in the distal direction.

As shown in FIGS. 15C, once distal ring 1513 is exposed, the distal ring can be partially released by detaching release wire 1519a from attachment point 1517a, allowing the distal ring to flip out into a position such that the face of the ring is no longer parallel to the central axis of transcatheter system 1501. Release wire 1519c maintains attachment to inner sheath 1509.

As shown in FIGS. 15C and 15D, transcatheter system 1501 is pulled proximately such that the distal end of outer sheath 1507 is pulled proximately back through aperture 1502. In addition, inner sheath 1509 is pulled proximately such that distal ring is pulled back into proximity and contact with the distal side of tissue wall 1500. A cinching cord 1521 is attached to distal ring 1513 and proximal ring 1515 that is still within outer sheath. As outer sheath 1507 and inner sheath 1509 are pulled proximately, cinching cord 1521 traverses through aperture 1502, maintaining the connection between distal ring 1513 and the proximal ring.

FIG. 15E depicts the exposure of proximal ring 1515. For illustrative purposes, distal ring 1513 is shown within the space on the distal side of tissue wall 1500, however, at this stage it is within proximity and/or in contact with the tissue wall as shown in FIG. 15D. Proximal ring 1515 is attached to inner sheath 1509 at attachment point 1517b via release wire 1519b. To expose proximal ring 1515, outer sheath 1507 can be slide in the proximal direction and/or inner sheath 1509 can be slide in the distal direction. Once proximal ring 1515 is exposed, the proximal ring is released by detaching release wire 1519b (FIG. 15F).

FIGS. 15G and 15H depict the localization of proximal ring 1515 and finalization of the installation of cinching ring system 1511. Proximal ring 1515 is aligned with distal ring 1513 utilizing one or more radiopaque markers 1523. Proximal ring 1515 is also localized to the proximal side of tissue wall 1500 via the cinching mechanism. The proximal ends (1521a and 1521b) of cinching cord 1521 are pulled in the proximal direction, resulting in tightening of the cinching cord and proximal ring being pulled in a distal direction toward tissue wall 1500. Once proximal ring 1515 comes into proximity and/or in contact with tissue wall 1500, the alignment of the proximal ring and distal ring 1513 can be assured and a final tightening of cord 1521 can be performed such that the rings are aligned with one another and secured to the tissue wall. Cinching cord 1521 can be cut and tied off or secured by any other means to keep the cord taut and cinching ring system 1511 in place via constrictive forces. Inner sheath 1509 and nose cone 1505 are pulled in the proximate direction through cinching ring system 1511 and aperture 1502, and transcatheter system 1501 is retracted and removed, leaving the cinching ring system installed.

In the final steps of installing a plug, the inner sheath and nose cone is pulled in the proximal direction back through an aperture or slit within a cover spanning the distal ring and/or a cover spanning a proximal ring. A final step of sealing the aperture or slit within the cover(s) can be performed, which can be done by various means (e.g., local stitching to close, cinching a cord to close, utilizing an adhesive to close).

### Examples of the Disclosure

1. An example system for cinching two rings together, comprising:
a first ring comprising an outer face, an inner face, and a plurality of stitch holes;
a second ring comprising an outer face, an inner face, and a plurality of stitch holes; and
a set of one or more cords that is stitched along and between the first ring and the second ring via the plurality of stitch holes of the first and second rings, wherein each of the two termini of each cord extends out from the external face of the first ring via a stitch hole of the first ring; and wherein the system has a cinching mechanism capability that moves the first ring and the second ring towards one another when the set of one or more cords is tightened by concurrently pulling the two termini of each cord in a direction away from the external face of the first ring.

2. The example system of 1 further comprising a means for providing a friction force against the first ring when tightening the cord by concurrently pulling the two termini of the cord in a direction away from the external face of the first ring.

3. The example system of 2, wherein the means for providing a friction force are a set of two tubes, each tube surrounding a portion of the cord that extends out from the external face of the first ring via a stitch hole of the first ring.

4. The example system of any one of 1 to 3 wherein the set of one or more cords is a single cord.

5. The example system of any one of 1 to 3 wherein the set of one or more cords is a plurality of cords.

6. An example cinch ring system for providing a shunt within the body of a patient, comprising:
a proximal ring comprising a proximal face, a distal face, and a plurality of stitch holes;
a distal ring comprising a proximal face, a distal face, and a plurality of stitch holes; and
a cord that is stitched along and between the proximal ring and the distal ring via the plurality of stitch holes of the proximal and distal rings, wherein each of the two termini of the cord extend out from the proximal face of the proximal ring via a stitch hole of the proximal ring; and wherein the system has a cinching mechanism capability that moves the proximal ring and the distal ring towards one another when the cord is tightened by concurrently pulling the two termini of the cord in a proximal direction away from the proximal face of the proximal ring.

7. The example system of 6, wherein the proximal ring or the distal ring contain at least one radiopaque marker.

8. The example system of 7, wherein the proximal ring and the distal ring each contain a plurality of radiopaque markers.

9. The example system of 8, the plurality of radiopaque markers of the proximal ring are in an orientation that mirrors the plurality of radiopaque markers of the distal ring when the proximal ring and the distal ring are provided in parallel planes.

10. The example system of 8, wherein the plurality of radiopaque markers of the proximal ring and the distal ring are each provided in an orientation relative to one another such that the opaque markers lack mirrored symmetry along one central axis of the ring.

11. The example system of any one of 6 to 10, wherein at least one ring of the proximal ring and the distal rings comprises a set of one or more protrusions extending outwardly from the ring.

12. The example system of any one of 6 to 11, wherein at least one ring of the proximal ring and the distal rings comprises a set of one or more windows.

13. The example system of any one of 6 to 12, wherein at least one stitch hole of the proximal ring or the distal ring incorporates a cord grip.

14. The example system of any one of 6 to 12, wherein each stitch hole of the proximal ring in which the two termini of the cord extend out from incorporates a cord grip.

15. The example system of any one of 6 to 14, wherein at least one ring of the proximal ring and the distal ring incorporates a sensor.

16. The example system of any one of 6 to 15, wherein the proximal ring and the distal ring are each provided in an elongated shape.

17. The example system of 6 to 16, wherein the proximal ring and the distal ring are each capable of folding or curving inward such that it is capable of being sheathed within a transcatheter capable of delivery via the circulatory system.

18. The example system of 17, wherein the proximal ring and the distal ring each have a central aperture with an area that is greater than the cross-sectional area of the transcatheter.

19. The example system of any one of 6 to 18, wherein the proximal ring and the distal ring are each composed of a biocompatible and malleable material.

20. The example system of any one of 6 to 19, wherein the cord is composed of a biocompatible material and is provided as a wire, a monofilament, or a twine.

21. An example cinch ring system for providing a plug within the body of a patient, comprising:
a proximal ring comprising a proximal face, a distal face, and a plurality of stitch holes;
a distal ring comprising a proximal face, a distal face, and a plurality of stitch holes, wherein a cover spans the central aperture of at least one ring of the proximal ring and the distal ring; and
a cord that is stitched along and between the proximal ring and the distal ring via the plurality of stitch holes of the proximal and distal rings, wherein each of the two termini of the cord extend out from the proximal face of the proximal ring via a stitch hole of the proximal ring; and wherein the system has a cinching mechanism capability that moves the proximal ring and the distal ring towards one another when the cord is tightened by concurrently pulling the two termini of the cord in a proximal direction away from the proximal face of the proximal ring.

22. The example system of 21, wherein a cover spans the central aperture of each ring of the proximal ring and the distal ring.

23. The example system of 21 or 22, wherein each cover incorporates a through hole or slit.

24. The example system of 23, wherein the through hole or slit is sealable.

25. The example system of any one of 21 to 24, wherein the proximal ring or the distal ring contain at least one radiopaque marker.

26. The example system of any one of 21 to 25, wherein at least one ring of the proximal ring and the distal rings comprises a set of one or more protrusions extending outwardly from the ring.

27. The example system of any one of 21 to 26, wherein the proximal ring and the distal ring are each provided in an elongated shape.

28. The example system of 21 to 27, wherein the proximal ring and the distal ring are each capable of folding in or curving in itself such that it is capable of being sheathed within a transcatheter capable of delivery via the circulatory system.

29. The example system of any one of 21 to 28, wherein the proximal ring and the distal ring are each composed of a biocompatible and malleable material.

30. The example system of any one of 21 to 29, wherein the cord is composed of a biocompatible material and is provided as a wire, a monofilament, or a twine.

31. An example delivery system for delivering a cinch ring system to a target site within the body of a patient, comprising:
a catheter comprising an outer sheath and an inner sheath; and
a cinch ring system comprising:
   a proximal ring comprising a proximal face, a distal face, and a plurality of stitch holes;
   a distal ring comprising a proximal face, a distal face, and a plurality of stitch holes; and
   a cord that is stitched along and between the proximal ring and the distal ring via the plurality of stitch holes of the proximal and distal rings;
   wherein the cinch ring system has a cinching mechanism;
wherein the cinch ring system is contained within the outer sheath and is in connection with the inner sheath of the catheter.

32. The example system of 31, wherein the proximal ring and the distal ring are each in a deliverable conformation within the outer sheath of the catheter.

33. The example system of 32, wherein the proximal ring and the distal ring are each an elongated shape that is folded or curved inwards.

34. The example system of 31, wherein the proximal ring and the distal ring are spaced apart from one another to facilitate traversing through the vasculature system of a patient.

35. The example system of any one of 31 to 34, wherein the cinch ring system is in connection with the inner sheath via a set of one or more release wires.

36. The example system of 35, wherein the set of one or more release wires comprises at least three release wires, wherein each release wire provides an attachment point to connect the cinch ring system with the inner sheath.

37. The example system of 36, wherein a first release wire provides a first attachment to the distal ring, wherein a second release wire provides an attachment to the proximal ring, and wherein a third release wire provides a second attachment to the distal ring.

38. The example system of any one of 31 to 37, wherein the inner sheath and the outer sheath are each independently capable of moving in a proximal direction or in a distal direction.

39. The example system of 38, wherein the connection of the cinch ring system to the inner sheath allows for the cinch ring system to be sheathed and unsheathed within the outer sheath.

40. The example system of 31, wherein a cover spans the central aperture of each ring of the proximal ring and the distal ring, wherein each cover incorporates a through hole or slit, and wherein the inner sheath traverses through each through hole or slit.

41. An example method of cinching a cinch ring system, the method comprising: providing a cinch ring system comprising:
a first ring comprising an outer face, an inner face, and a plurality of stitch holes;
a second ring comprising an outer face, an inner face, and a plurality of stitch holes; and
a cord that is stitched along and between the first ring and the second ring via the plurality of stitch holes of the first and second rings, wherein each of the two termini of the cord extend out from the external face of the first ring via a stitch hole of the first ring;
situating the cinch ring system such that the inner face of the first ring is facing the inner face of the second ring with a central space between the first ring and second ring;
pulling on the two termini of the cord concurrently, tightening the cord such that the central space between the first ring and the second ring is narrowed.

42. The example method of 41, wherein a means for providing a friction force is utilized to assist the pulling on the two termini of the cord concurrently to tighten the cord.

43. The example method of 41, wherein the means for providing a friction force are a set of two tubes, each tube surrounding a portion of the cord that extends out from the external face of the first ring via a stitch hole of the first ring.

44. The example method of 41, 42, or 43 further comprising securing the cord to keep the cord taut and the cinching ring system in place via constrictive forces.

45. The example method of 44, wherein the cord is secured by tying off the cord.

46. An example method of delivering and installing a cinch ring system within the body of a patient, comprising:
providing a transcatheter delivery system comprising:
   a catheter comprising an outer sheath and an inner sheath; and
   a cinch ring system comprising:
      a proximal ring comprising a proximal face, a distal face, and a plurality of stitch holes;
      a distal ring comprising a proximal face, a distal face, and a plurality of stitch holes; and
      a cord that is stitched along and between the proximal ring and the distal ring via the plurality of stitch holes of the proximal and distal rings, wherein each of the two termini of the cord extend out from the proximal face of the proximal ring via a stitch hole of the proximal ring;
      wherein the cinch ring system is contained within the outer sheath and is in connection with the inner sheath of the catheter; delivering the transcatheter delivery system to a tissue wall having an aperture; and installing the cinch ring system to the tissue wall.

47. The example method of 46, wherein the installing of the cinch ring system to the tissue wall comprises:
releasing the distal ring on the distal side of the tissue wall;
releasing the proximal ring on the proximal side of the tissue wall, wherein at least a portion of the cord that is stitched between the distal ring and proximal ring traverses through the aperture of the tissue wall; and
cinching the cinch ring system such that the distal ring is in proximity to or in contact with the distal side of the tissue wall and the proximal ring is in proximity to or in contact with the proximal side of the tissue wall.

48. The example method of 47, wherein the cinch ring system is in connection with the inner sheath via a set of one or more release wires, wherein at least one release wire of the set is in connection with the distal ring, and wherein the releasing of the distal ring on the distal side of the tissue wall comprises disconnecting the at least one release wire of the set that is in connection with the distal ring.

49. The example method of 47, wherein the cinch ring system is in connection with the inner sheath via a set of one or more release wires, wherein at least one release wire of the set is in connection with the proximal ring, and wherein the releasing of the proximal ring on the proximal side of the tissue wall comprises disconnecting the at least one release wire of the set that is in connection with the proximal ring.

50. The example method of 47, wherein the cinching of the cinch ring system comprises pulling on the two termini of the cord in a proximal direction to tighten the cord.

51. The example method of 47, wherein the installing of the cinch ring system to the tissue wall further comprises securing the cord to keep the cord taut and the cinching ring system in place via constrictive forces.

52. The example method of 51, wherein the cord is secured using a cord grip.

53. The example method of 51 or 52, wherein the cord is secured by tying off the cord.

54. The example method of 46, wherein the proximal ring and the distal ring each contain a plurality of radiopaque markers; the method further comprising:
aligning the proximal ring and the distal ring via the plurality of radiopaque markers on each the proximal ring and the distal ring.

55. The example method of 54, wherein the plurality of radiopaque markers of the proximal ring are in an orientation that mirrors the plurality of radiopaque markers of the distal ring when the cinch ring system is installed on the tissue wall.

56. The example method of 46, wherein the proximal ring and the distal ring are each folded or curved inward when within the outer sheath.

57. The example method of 46, wherein installation of the cinch ring system to the tissue wall provides a shunt via the aperture in the tissue wall.

58. The example method of 46, wherein a cover spans a central aperture of at least one ring of the proximal ring and the distal ring, wherein installation of the cinch ring system to the tissue wall provides a plug of the aperture in the tissue wall.

59. The example method of 58, wherein the installation of the cinch ring system to the tissue wall further comprises sealing a through hole or slit in the cover.

60. The example method as in any one of 46 to 59, wherein delivering of the transcatheter delivery system is delivered via a transfemoral approach, a subclavian approach, a transapical approach, or a transaortic approach.

61. An example method of installing a cinch ring system on a nonliving simulation having a simulated tissue wall, comprising:
providing a nonliving tissue wall having an aperture, wherein the nonliving tissue wall provides a simulation of a tissue wall within a patient;
providing a transcatheter delivery system comprising:
   a catheter comprising an outer sheath and an inner sheath; and
   a cinch ring system comprising:
      a proximal ring comprising a proximal face, a distal face, and a plurality of stitch holes;
      a distal ring comprising a proximal face, a distal face, and a plurality of stitch holes; and
      a cord that is stitched along and between the proximal ring and the distal ring via the plurality of stitch holes of the proximal and distal rings, wherein each of the two termini of the cord extend out from the proximal face of the proximal ring via a stitch hole of the proximal ring;
      wherein the cinch ring system is contained within the outer sheath and is attached to the inner sheath of the catheter;
releasing the distal ring on the distal side of the nonliving tissue wall;
releasing the proximal ring on the proximal side of the nonliving tissue wall, wherein at least a portion of the cord stitched between the distal ring and proximal ring traverses through the aperture of the nonliving tissue wall;
pulling on the two termini of the cord in a proximal direction to tighten the cord such that the distal ring is in proximity to or in contact with the distal side of the nonliving tissue wall and the proximal ring is in proximity to or in contact with the proximal side of the nonliving tissue wall.

62. An example cinch ring system for installing a device within the body of a patient, comprising:
a proximal ring comprising a proximal face, a distal face, a central aperture, and a plurality of stitch holes;
a distal ring comprising a proximal face, a distal face, a central aperture, and a plurality of stitch holes;
a cord that is stitched along and between the proximal ring and the distal ring via the plurality of stitch holes of the proximal and distal rings, wherein each of the two termini of the cord extend out from the proximal face of the proximal ring via a stitch hole of the proximal ring; and wherein the system has a cinching mechanism capability that moves the proximal ring and the distal ring towards one another when the cord is tightened by concurrently pulling the two termini of the cord in a proximal direction away from the proximal face of the proximal ring;
a device having a body that is in connection with the central aperture of the proximal ring or the central aperture of the distal ring; wherein the proximal ring or the distal ring is capable of sliding along the body the device when the cinching mechanism is utilized.

63. The example system of 62, wherein the device is a sensor.

64. The example system of 63, wherein the sensor is a telemetric sensor.

65. The example system of 63, wherein the sensor is a hemodynamic sensor.

66. The example system of 63, wherein the sensor is a pressure sensor, a temperature sensor, a flow rate sensor, an oxygen saturation sensor, or a loop recorder.

67. The example system of any one of 62 to 66, wherein at least one ring of the proximal ring and the distal rings comprises a set of one or more protrusions extending outwardly from the ring.

68. The example system of 67, wherein each protrusion of the set one or more protrusions has length between 1X and 5X of the internal diameter of proximal ring or the distal ring.

69. The example systems of anyone of 62 to 68, wherein the proximal ring or the distal ring contain at least one radiopaque marker.

70. The example system of any one of 62 to 69, wherein at least one ring of the proximal ring and the distal rings comprises a set of one or more windows.

71. The example system of any one of 62 to 70, wherein at least one stitch hole of the proximal ring or the distal ring incorporates a cord grip.

72. The example system of any one of 62 to 70, wherein each stitch hole of the proximal ring in which the two termini of the cord extend out from incorporates a cord grip.

73. The example system of 62 to 72, wherein the proximal ring and the distal ring have a total area that is less than a cross-sectional area of a transcatheter, wherein in the total area is exclusive of any extended protrusions.

74. The example system of 73, wherein the proximal ring and the distal ring each have a set of one or more extended protrusions, wherein the extended protrusions are capable of being folded into a position perpendicular to the faces of the proximal ring and the distal ring such that the proximal ring and the distal ring are each capable of fitting within the cross-section area of the transcatheter.

75. The example system of any one of 62 to 74, wherein the proximal ring and the distal ring are each composed of a biocompatible and malleable material.

76. The example system of any one of 62 to 75, wherein the cord is composed of a biocompatible material and is provided as a wire, a monofilament, or a twine.

### DOCTRINE OF EQUIVALENTS

While the above description contains many specific examples, these should not be construed as limitations on the scope of the disclosure provided, but rather as examples to illustrate the innovative concepts to be claimed. Accordingly, the scope of the disclosure should be determined not by the examples illustrated, but by the appended claims and their equivalents.

What is claimed is:

1. A cinch ring system for providing a shunt or a plug within the body of a patient, comprising:
a proximal ring comprising a proximal face, a distal face, and a plurality of stitch holes;
a distal ring comprising a proximal face, a distal face, and a plurality of stitch holes; and
a cord that is stitched along and between the proximal ring and the distal ring via the plurality of stitch holes of the proximal and distal rings, wherein each of the two termini of the cord extend out from the proximal face of the proximal ring via a stitch hole of the proximal ring;
and wherein the system has a cinching mechanism capability that moves the proximal ring and the distal ring towards one another when the cord is tightened by concurrently pulling the two termini of the cord in a proximal direction away from the proximal face of the proximal ring.

2. The system of claim 1, wherein the proximal ring or the distal ring contain at least one radiopaque marker.

3. The system of claim 1, wherein at least one ring of the proximal ring and the distal rings comprises a set of one or more protrusions extending outwardly from the ring.

4. The system of claim 1, wherein at least one stitch hole of the proximal ring or the distal ring incorporates a cord grip.

5. The system of claim 1, wherein at least one ring of the proximal ring and the distal ring incorporates a sensor.

6. The system of claim 1, wherein the proximal ring and the distal ring are each capable of being sheathed within a transcatheter capable of delivery via the circulatory system.

7. The system of claim 1, wherein the proximal ring and the distal ring are each composed of a biocompatible and malleable material.

8. A cinch ring system for providing a plug within the body of a patient, comprising:
a proximal ring comprising a proximal face, a distal face, and a plurality of stitch holes;
a distal ring comprising a proximal face, a distal face, and a plurality of stitch holes, wherein a cover spans the central aperture of at least one ring of the proximal ring and the distal ring; and
a cord that is stitched along and between the proximal ring and the distal ring via the plurality of stitch holes of the proximal and distal rings, wherein each of the two termini of the cord extend out from the proximal face of the proximal ring via a stitch hole of the proximal ring;
and wherein the system has a cinching mechanism capability that moves the proximal ring and the distal ring towards one another when the cord is tightened by concurrently pulling the two termini of the cord in a proximal direction away from the proximal face of the proximal ring.

9. The system of claim 8, wherein a cover spans the central aperture of each ring of the proximal ring and the distal ring.

10. The system of claim 8, wherein each cover incorporates a through hole or slit.

11. The system of claim 10, wherein the through hole or slit is sealable.

12. The system of claim 8, wherein the proximal ring or the distal ring contain at least one radiopaque marker.

13. The system of claim 8, wherein at least one ring of the proximal ring and the distal rings comprises a set of one or more protrusions extending outwardly from the ring.

14. The system of claim 8, wherein the proximal ring and the distal ring are each capable of being sheathed within a transcatheter capable of delivery via the circulatory system.

15. The system of claim 8, wherein the proximal ring and the distal ring are each composed of a biocompatible and malleable material.

16. A delivery system for delivering a cinch ring system to a target site within the body of a patient, comprising:
a catheter comprising an outer sheath and an inner sheath; and
a cinch ring system comprising:
a proximal ring comprising a proximal face, a distal face, and a plurality of stitch holes;
a distal ring comprising a proximal face, a distal face, and a plurality of stitch holes; and
a cord that is stitched along and between the proximal ring and the distal ring via the plurality of stitch holes of the proximal and distal rings;
wherein the cinch ring system has a cinching mechanism;
wherein the cinch ring system is contained within the outer sheath and is in connection with the inner sheath of the catheter.

17. The system of claim 16, wherein the cinch ring system is in connection with the inner sheath via a set of one or more release wires.

18. The system of claim 17, wherein the set of one or more release wires comprises at least three release wires, wherein each release wire provides an attachment point to connect the cinch ring system with the inner sheath.

19. The system of claim 18, wherein a first release wire provides a first attachment to the distal ring, wherein a second release wire provides an attachment to the proximal ring, and wherein a third release wire provides a second attachment to the distal ring.

20. The system of claim 16, wherein a cover spans the central aperture of each ring of the proximal ring and the distal ring, wherein each cover incorporates a through hole or slit, and wherein the inner sheath traverses through each through hole or slit.
